(19) United States
(12) Patent Application Publication
(10) Pub. No.: US 2026/0047797 A1
(43) Pub. Date: Feb. 19, 2026

(54) PHYSIOLOGICAL METRICS FOR DETERMINING STROKE RISK

(71) Applicants: California Institute of Technology, Pasadena, CA (US); University of Southern California, Los Angeles, CA (US)

(72) Inventors: Simon Mahler, Marseille (FR); Yu Xi Huang, South Pasadena, CA (US); Changhuei Yang, South Pasadena, CA (US); Charles Liu, San Marino, CA (US)

(73) Assignees: California Institute of Technology, Pasadena, CA (US); University of Southern California, Los Angeles, CA (US)

(21) Appl. No.: 19/334,485

(22) Filed: Sep. 19, 2025

Related U.S. Application Data
(63) Continuation-in-part of application No. 18/934,954, filed on Nov. 1, 2024.
(60) Provisional application No. 63/547,033, filed on Nov. 2, 2023.
(60) Provisional application No. 63/696,966, filed on Sep. 20, 2024.
(60) Provisional application No. 63/713,761, filed on Oct. 30, 2024.

(51) Int. Cl. A61B 5/00; A61B 5/0205; A61B 5/026; A61B 5/1455

(52) U.S. Cl. CPC A61B 5/4064; A61B 5/0205; A61B 5/4884; A61B 5/6803; A61B 5/7267; A61B 5/7275; A61B 5/0261; A61B 5/14553

(57) ABSTRACT

Techniques for determining stroke risk are provided. In some embodiments, the techniques may involve causing, using one or more light sources disposed on a headset worn by a user, light to be emitted into a head of the user, obtaining, using one or more light detectors disposed on the headset, information indicative of light reflected from one more structures within the head of the user, wherein at least a portion of the obtained information is obtained during a time period of hypercapnic stimulation, based on the obtained information, determining a representation of one or more cerebral blood metrics, providing the representation of the one or more cerebral blood metrics as input to a computational model, and determining a stroke risk score based on output of the computational model.

### CROSS-REFERENCES TO RELATED APPLICATIONS

This application claims priority to and benefit of U.S. Provisional Patent Application No. 63/696,966, filed on Sep. 20, 2024, and titled “Stroke Risk Assessment By Measuring Blood Flow-To-Volume Ratio Changes With Laser Speckle Imaging Device” and to U.S. Provisional Patent Application No. 63/713,761, filed on Oct. 30, 2024, and titled “Optical Brain Blood Dynamics Tracing for Stroke Risk Assessment and Prevention;” this application is a continuation-in-part of U.S. patent application Ser. No. 18/934,954, filed on Nov. 1, 2024, and titled “PHYSIOLOGICAL METRICS FOR DETERMINING STROKE RISK,” which claims priority to and benefit of U.S. Provisional Patent Application No. 63/547,033, filed on Nov. 2, 2023 and titled “Cerebral Perfusion Reserve Stroke Risk Scoring Through Comprehensive Brain Blood Dynamic Tracing;” all of these applications are incorporated by reference herein in their entireties and for all purposes.

### FEDERALLY SPONSORED RESEARCH OR DEVELOPMENT

This invention was made with government support under Grant No. EY033086 awarded by the National Institutes of Health. The government has certain rights in the invention.

### FIELD

Certain aspects generally pertain to techniques for assessing stroke risk using physiological metric data.

### BACKGROUND

Stroke is a global health concern, with a distressingly high incidence, and substantial morbidity and mortality. Annually, more than ten million people worldwide are impacted by strokes, imposing a heavy toll on affected individuals and their families, with significant health, financial, and quality of life burdens. In the United States alone, strokes affect nearly 800,000 individuals each year. Identifying patients likely to experience stroke may aid in prescribing and/or monitoring lifestyle changes or medications that decrease likelihood of stroke. However, identifying patients likely to experience stroke or determining a patient's stroke risk is difficult or impossible.

### SUMMARY

Techniques disclosed herein may be practiced with a processor-implemented method, a system comprising one or more processors and one or more processor-readable media, and/or one or more non-transitory processor-readable media.

According to some embodiments, a method for determining stoke risk may involve causing, using one or more light sources disposed on a headset worn by a user, light to be emitted into the head of the user. The method may further involve obtaining, using one or more light detectors disposed on the headset, information indicative of light reflected from one more structures within the head of the user, wherein a portion of the obtained information spans a time period during which the user was holding their breath. The method may further involve based on the obtained information, determining one or more cerebral blood metrics. The method may further involve providing a representation of the one or more cerebral blood metrics as input to a trained machine learning model. The method may further involve determining a likelihood the user will experience a stroke over a predetermined future time period based on an output of the trained machine learning model.

According to some embodiments, a system for determining stroke risk may comprise: a headband configured to encircle a head of a wearer of a headset; a plurality of light sources attached to the headband; a plurality of light detectors attached to the headband; and one or more processors. The one or more processors may be configured to: cause, using the one or more light sources, light to be emitted into the head of the wearer; obtain, using the one or more light detectors, information indicative of light reflected from one more structures within the head of the wearer, wherein a portion of the obtained information spans a time period during which the wearer was holding their breath; based on the obtained information, determine one or more cerebral blood metrics; and determine a likelihood the wearer will experience a stroke over a predetermined future time period based on the one or more cerebral blood metrics.

According to some embodiments, a method for determining stroke risk may involve causing, using one or more light sources disposed on a headset worn by a user, light to be emitted into the head of the user. The method may further involve obtaining, using one or more light detectors disposed on the headset, information indicative of light reflected from one more structures within the head of the user, wherein a portion of the obtained information spans a time period during which the user was holding their breath. The method may further involve based on the obtained information, determining a cerebral blood flow as a function of time and a cerebral blood volume as a function of time, wherein the cerebral blood flow and the cerebral blood volume include the time period during which the user was holding their breath and a baseline time period before the user was holding their breath. The method may further involve determining a likelihood the user will experience a stroke over a predetermined future time period based on the cerebral blood flow and the cerebral blood volume.

According to some embodiments, a method of training a machine learning model to predict stroke risk may involve obtaining training data, the training data comprising, for a group of users, representations of cerebral blood metrics, wherein for each training sample, a portion of the obtained data spans a time period during which the user was holding their breath, and wherein each training sample includes a corresponding ground truth stroke risk for the user. The method may further involve providing the training data to a machine learning model, wherein the machine learning model takes, as input, the representations of the cerebral blood metrics and generates, as an output, a prediction of stroke risk. The method may further involve updating the machine learning model based on differences between the ground truth stroke risk and the predicted stroke risk to generate a trained machine learning model configured to predict stroke risk.

Some embodiments pertain to methods for determining stroke risk. In some cases, methods cause using one or more light sources disposed on a headset worn by a user, light to be emitted into a head of the user. These methods also obtain, using one or more light detectors disposed on the headset, information indicative of light reflected from one more structures within the head of the user, wherein at least a portion of the obtained information is obtained during a time period of hypercapnic stimulation. These methods also based on the obtained information, determine a representation of one or more cerebral blood metrics, provide the representation of the one or more cerebral blood metrics as input to a computational model (e.g., machine learning model), and determine a stroke risk score based on output of the computational model.

Some embodiments pertain to apparatus for determining stroke risk. In some cases, an apparatus includes a headband configured to attach to a head of a user during operation, a plurality of light sources attached to the headband, a plurality of light detectors attached to the headband, and one or more processors. The one or more processors are configured to, during operation, cause, using the plurality of light sources, light to be emitted into a head of the user, obtain, using the plurality of light detectors, information indicative of light reflected from one more structures within the head of the user, wherein at least a portion of the obtained information is obtained during a time period of hypercapnic stimulation, based on the obtained information, determine a stroke risk score, and display the stroke risk score.

These and other features are described in more detail below with reference to the associated drawings.

### BRIEF DESCRIPTION OF THE DRAWINGS

FIG. 1 is a schematic diagram of components of a system that generates stroke likelihood data in accordance with some embodiments.

FIGS. 2A and 2B are diagrams illustrating an example headset in accordance with some embodiments.

FIG. 3 is a diagram illustrating example light paths through the skull and brain of a patient in accordance with some embodiments.

FIG. 4 is a schematic illustration of a front view of components of an apparatus for generating cerebral blood metrics data, according to some embodiments.

FIG. 5 is a schematic illustration of a top view of the apparatus in FIG. 4 illustrating example light paths through the skull and a portion of a brain of a user, according to some embodiments.

FIG. 6 illustrates graphs depicting example techniques to determine cerebral blood flow in accordance with some embodiments.

FIG. 7 illustrates example cerebral blood volume, cerebral blood flow, and cerebral blood oxygen during a breath holding task in accordance with some embodiments.

FIG. 8 illustrates example graphs of cerebral blood flow and cerebral blood volume during a breath holding task in accordance with some embodiments.

FIG. 9 is a flowchart of an example process for determining stroke likelihood data in accordance with some embodiments.

FIG. 10 is a flowchart depicting an example process for training a machine learning model to predict a stroke likelihood in accordance with some embodiments.

FIGS. 11A and 11B depict example experimental data in accordance with some embodiments.

FIG. 12 is a table listing example features extracted from experimental physiological metric(s) data, according to various embodiments.

FIG. 13 is a schematic diagram of components of a system that generates stroke likelihood data in accordance with some embodiments.

FIG. 14 is a flowchart of an example process for determining a stroke likelihood using cerebral blood metrics in accordance with some embodiments.

FIG. 15 is a flowchart of an example process for training a machine learning model in accordance with some embodiments.

FIG. 16 is a graph depicting example experimental data in accordance with some embodiments.

FIG. 17 is a flowchart of an example process for determining a stroke risk score using cerebral blood metrics in accordance with some embodiments.

FIG. 18 is a flowchart of an example process for training a machine learning model in accordance with some embodiments.

FIG. 19 is a diagram depicting an example of a process for determining a stroke risk score, according to some embodiments.

FIG. 20 is a diagram depicting an example of a process for training and testing one or more machine learning models that generate stroke risk scores, according to some embodiments.

FIG. 21 is a block diagram depicting an example computing device, according to certain embodiments.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

Stroke is a global health concern, with a distressingly high incidence, and substantial morbidity and mortality. Annually, more than ten million people worldwide are impacted by strokes, imposing a heavy toll on affected individuals and their families, with significant health, financial, and quality of life burdens. In the United States alone, strokes affect nearly 800,000 individuals each year. Identifying patients likely to experience stroke may aid in prescribing and/or monitoring lifestyle changes or medications that decrease likelihood of stroke. However, identifying patients likely to experience stroke or determining a patient's stroke risk is difficult or impossible using conventional techniques. In particular, conventional techniques typically utilize a questionnaire that evaluates health factors, such as hypertension, cholesterol levels, diabetes, and atrial fibrillation, etc. along with lifestyle-related factors such as body mass index, exposure to air pollution, smoking, unhealthy diet, alcohol consumption, and low physical activity, and the like. These risk profiles are based on population-level data and may be useful for a general assessment. However, such questionnaires are not definitive for determining the need for invasive or non-invasive evaluations, nor for guiding surgical or pharmacological interventions. For example, such questionnaires may give rise to a stroke risk prediction that is too general, and therefor, inaccurate. Moreover, use of such questionnaires may not accurately and precisely indicate whether various interventions (whether surgical, pharmacological, or behavioral) have modified a given patient's stroke risk, e.g., to determine whether a given intervention has lowered the patient's risk of stroke.

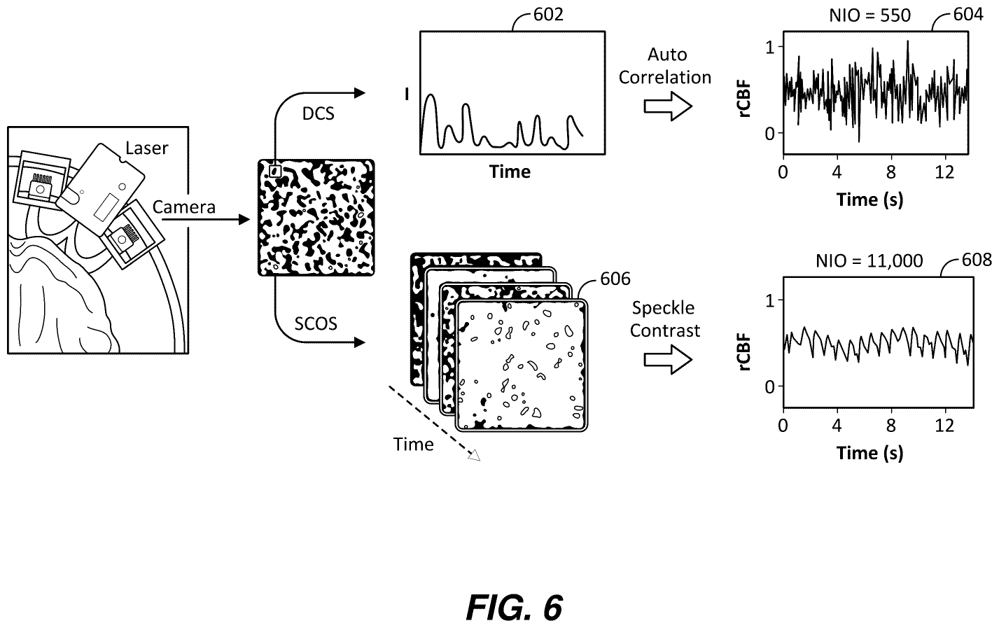

Disclosed herein are techniques for utilizing physiological metrics to predict stroke risk. In particular, certain techniques disclosed herein utilize cerebral perfusion metrics, which may include cerebral blood volume (CBV), cerebral blood flow (CBF), and/or cerebral blood oxygenation (CBO), to predict stroke risk. The cerebral perfusion metrics, sometimes referred to herein as “cerebral blood metrics,” may be obtained during a period of time of a brain stress test that elevates carbon dioxide levels in the bloodstream. This hypercapnic time period may include a time a patient or other user is holding their breath, time a user is administered a gas mixture with a predefined concentration of carbon dioxide (e.g., CO₂ concentration less than 5%, CO₂ concentration between stimulation 1-5%, etc.), rebreathing period where the patient breathes into a closed or partially closed system, voluntary hypoventilation, where the patient reduces breathing frequency or depth, or with the use of respiratory device, or another time during which conditions may stimulate hypercapnia or breath-retention in the user. Changes in the cerebral blood metrics during the hypercapnic stimulation time period relative to a baseline time period, and/or changes in the cerebral blood metrics in the time period after the hypercapnic stimulation time period may be utilized to predict stroke risk during a future time period. Because patients with a relatively high stroke risk have stiffer blood vessels, their blood vessels may be relatively harder to dilate. Accordingly, patients with a relatively high stroke risk may be observed to have relatively larger increases in cerebral blood flow and relatively lower changes in cerebral blood volume relative to patients with a low stroke risk. Other changes, such as slope of change in cerebral blood metrics, timing characteristics of the return to baseline of cerebral blood metrics after breath-holding or inhaling gas mixture with predefined CO₂ concentration terminates, etc. may be characterized. In some embodiments, a trained machine learning model may be provided with either extracted features of cerebral blood flow metrics, or with raw cerebral blood flow metrics data, and may generate a stroke prediction risk based on the cerebral blood flow metrics. In other embodiments, other types of computational models may be utilized. Cerebral blood metrics may be determined using data characterizing light reflected from various structures within a brain of a patient. For example, in some implementations, cerebral blood volume and/or cerebral blood flow metrics may be determined by transmitting light in an infrared wavelength range into the brain of the patient and measuring reflected light. In some implementations, cerebral blood flow may be determined using speckle contrast optical spectroscopy (SCOS), described below in more detail in connection with FIG. 6. In some embodiments, cerebral blood oxygenation may be determined by transmitting light at two wavelengths and measuring reflected/absorbed light at the two wavelengths. The two wavelengths may include an infrared wavelength and a near infrared wavelength.

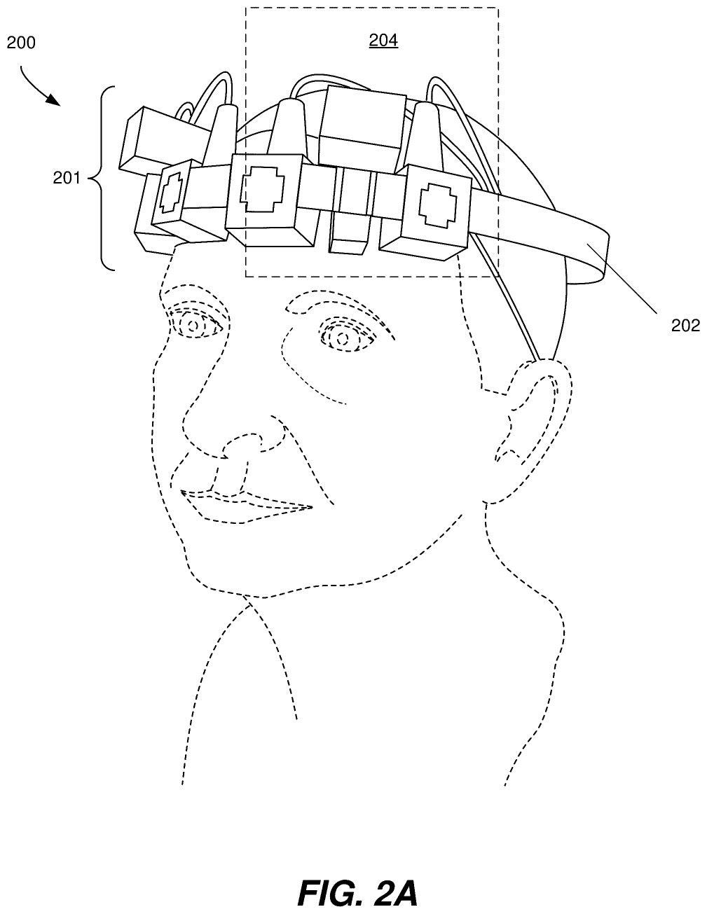

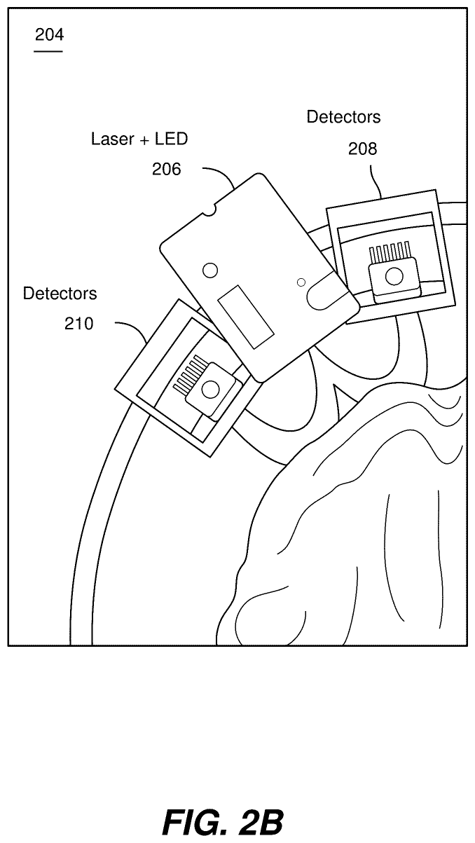

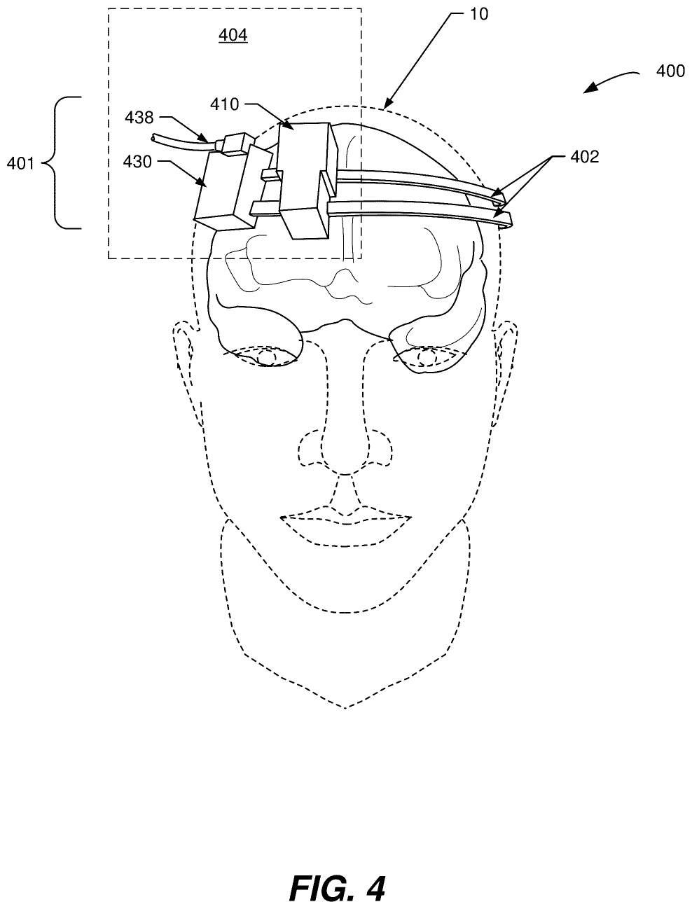

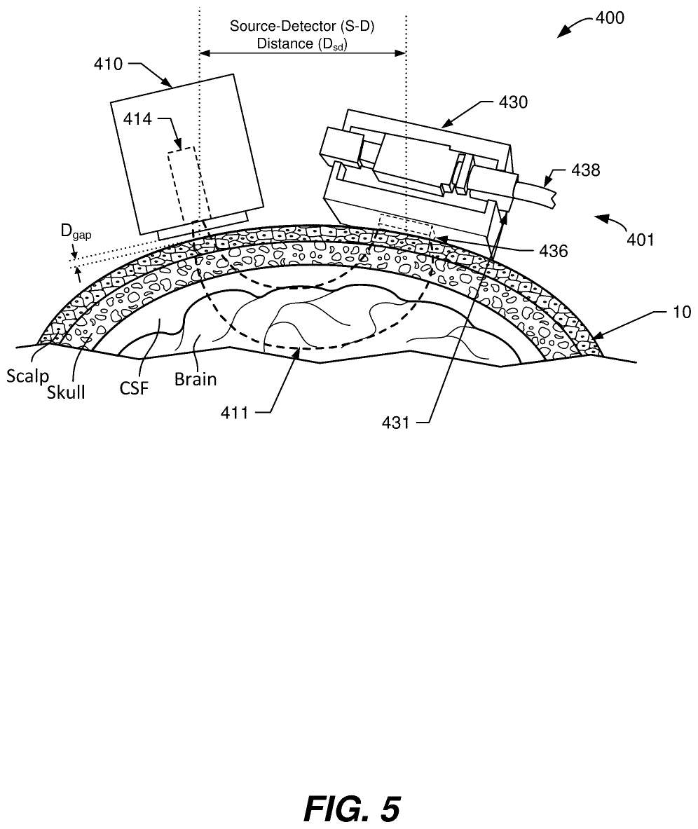

In some implementations, light may be transmitted into the brain of the patient using a light emitter, which may be a laser, a light emitting diode (LED), or the like. Scattered light may be captured using a light detector, which may be a camera, a photodetector, a single-photon avalanche diode (SPAD), a SPAD array, or the like. In instances in which cerebral blood oxygenation metrics are determined and therefore, in which light is transmitted at two wavelengths, two light sources (e.g., a laser and an LED) may be used to transmit light, and two detectors (e.g., a camera and a photodetector) may be used to capture reflected light. In some embodiments, two (or more) light sources may be packaged as one light emission package, and two (or more) detectors may be packaged as one light detection package. In some implementations, light sources and/or light detectors may be disposed on a headband configured to encircle the head of the patient (e.g., around the forehead). In some implementations, light sources and light detectors (or light emission packages and light detection packages) may be affixed to the headband at different locations. For example, the light sources and/or light emission packages may be affixed to the headband at positions corresponding to a forehead of the patient, the parietal lobe of the patient, the frontal lobe of the patient, etc. In some implementations, a light source and light detector may be fiber free in that there is no fiber coupling to either the light source or the light detector. Accordingly, all components of the headset may be head-mounted, which may reduce noise emanating from optical fibers' movement. In some implementations, the laser and the camera/detector may be placed directly atop the user's skin. An example of a headset is shown in and described below in connection with FIGS. 2A and 2B. Another example of a headset is shown in and described below in connection with FIGS. 4 and 5.

In various embodiments, systems or apparatus for determining stroke risk may employ a stroke prediction engine that can take, as input, hypercapnic stimulation data (e.g., breath-holding data or inhaling CO₂ gas mixture data), and generate, as an output, stroke likelihood data. In certain cases, the systems or apparatus for determining stroke risk can output stroke likelihood data in the form of an integer on a discrete scale (sometimes referred to herein as a risk score). For example, a system or apparatus for determining stroke risk may output an integer on a scale from 1 to 10, an integer on a scale from 1 to 100, an integer on a scale from 1 to 7, etc. In other cases, a system or apparatus for determining stroke risk may output a number on a continuous scale (e.g., a probability value that is a continuous number between 0 and 1). The stroke likelihood data may represent a likelihood that, given the physiological metrics represented in the hypercapnic stimulation data, the patient will have a stroke within a predetermined future time period (e.g., within the next year, within the next five years, within the lifetime of the patient, etc.).

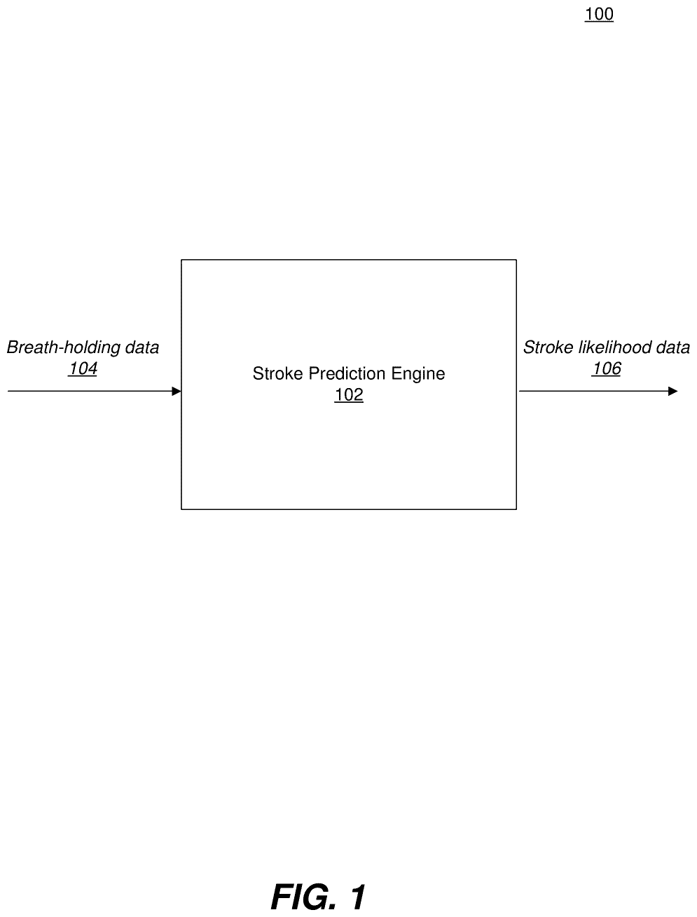

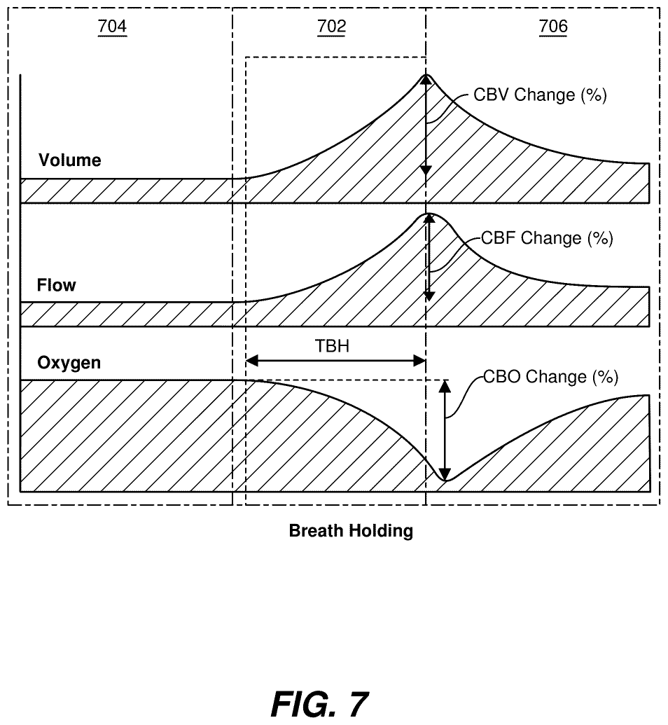

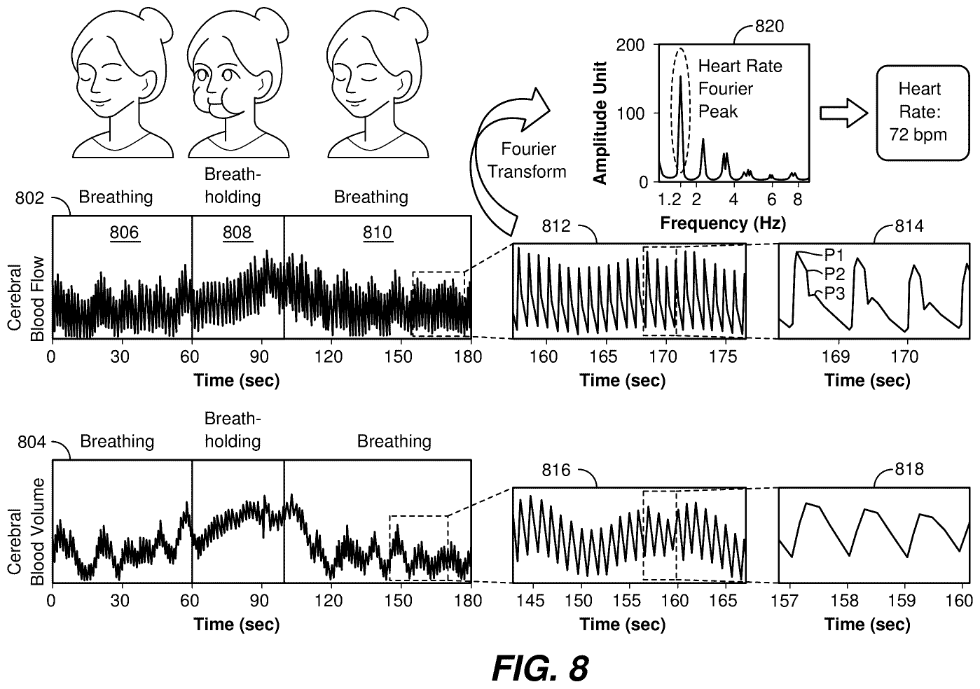

FIG. 1 is a block diagram of an example system 100 for determining stroke likelihood data in accordance with some embodiments. As illustrated, the system 100 includes a stroke prediction engine 102. In some implementations, stroke prediction engine 102 may include a trained machine learning model or other computational model configured to take, as input, breath-holding data 104, and generate, as an output, stroke likelihood data 106. The trained machine learning model may be a perceptron, a random forest, a deep neural network (DNN), or any other suitable architecture. In instances in which breath-holding data 104 includes raw cerebral blood metric data (e.g., traces of cerebral blood flow, cerebral blood volume, and/or cerebral blood oxygenation as a function of time, as shown in and described below in connection with FIG. 7), the trained machine learning model may be a DNN. Such a DNN may be able to identify features in the cerebral blood metric data not observable or identifiable by a human that are useful for predicting stroke risk. In other embodiments, in instances in which breath-holding data 104 includes extracted features of cerebral blood metric data, (as described in more detail in connection with FIGS. 7 and 8) the trained machine learning model may be a perceptron, a random forest, or other type of architecture configured to take extracted features as input and generate a stroke likelihood. Note that the breath-holding data may be obtained using light emitted into the brain of the patient using one or more light sources and using data representative of absorbed/reflected light using one or more light detectors. Such light emitters and light detectors may be disposed on a headband or headset, as shown in and described in more detail in connection with FIGS. 2A and 2B and FIG. 4.

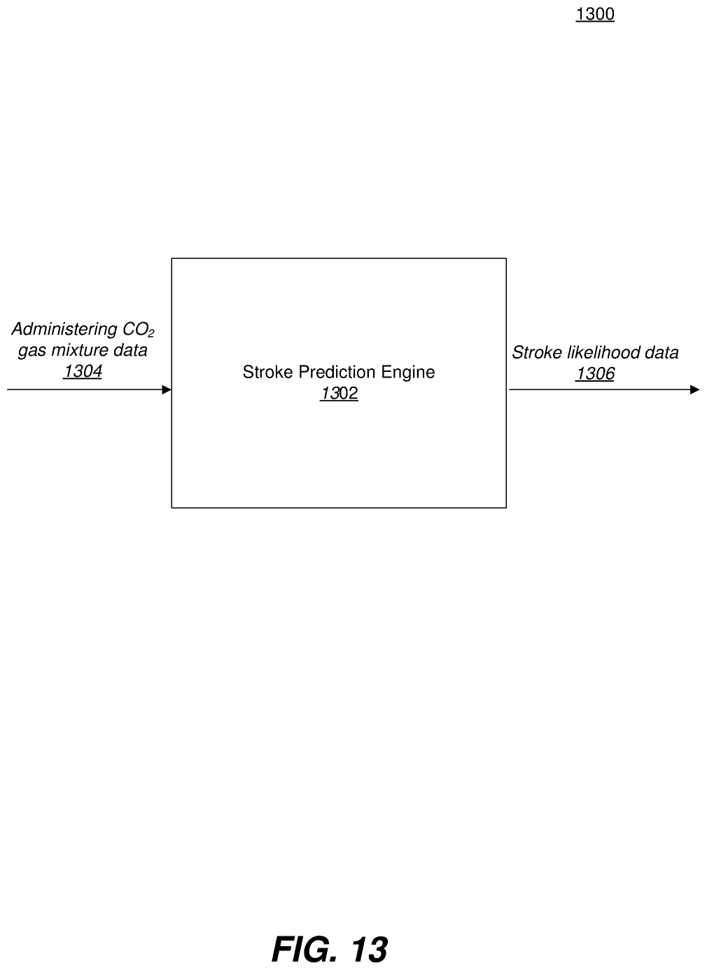

In accordance with other embodiments, a system for determining stroke likelihood data may include a trained machine learning model or other computational model configured to take, as input, inhaling air with low CO₂ concentration data or other brain stress test data, and generate, as an output, stroke likelihood data. An example system of such an embodiment is described in and with connection with FIG. 13.

In various implementations, a stroke prediction engine includes a trained machine learning model configured to take, as input, brain stress test data such as breath-holding data or hypercapnic stimulation data, and generate, as an output, stroke likelihood data. In other implementations, a stroke prediction engine may include another type of computational model configured to take, as input, breath-holding data or other physiological metric(s) data, and generate, as output stroke likelihood data. Some examples of other types of computational models that may be used include machine learning, deep neural networks, Bayesian networks, support vector machines or other kernel-based methods capable of handling physiological inputs, and hybrid models that combine physiological modeling (e.g. extracted features) with data-driven learning approaches.

Stroke likelihood data 106 may be a number on a discrete scale (e.g., an integer between 1 and 5), a number on a continuous scale (e.g., a probability value that is a continuous number between 0 and 1), or the like. Stroke likelihood data 106 may represent a likelihood that, given the cerebral blood metrics represented in breath-holding data 104, the patient will have a stroke within a predetermined future time period (e.g., within the next year, within the next five years, within the lifetime of the patient, etc.).

It should be noted that stroke prediction engine 102 may be implemented by one or more computing devices or one or more processors. For example, such a computing device and/or processor may be configured to analyze data from one or more light detectors, generate cerebral blood flow metrics, and/or provide data representative of the cerebral blood flow metrics to a trained machine learning model to generate stroke likelihood data 106. In some implementations, the one or more computing device and/or one or more processors may be disposed on the same headset or headband as the one or more light emitters and one or more light detectors. Additionally, or alternatively, the one or more computing devices and/or one or more processors may be communicatively coupled to the headset or headband, e.g., by a wireless or wired communication channel.

As described above, one or more light emitters and one or more light detectors may be disposed on a head-worn device by a patient. The light may be emitted toward and/or into the head of the patient, thereby probing cerebral blood flow. Reflected light may be captured by the one or more light detectors to assess and/or characterize absorbed and reflected light by structures internal to the patient's brain. In some implementations, a “channel” may be characterized by a light emission package and a corresponding light detector package. In some embodiments, a light emission package may include multiple light emitters, such as a laser and an LED. For example, a laser may emit light in a near infrared or infrared wavelength, and an LED may emit light in a visible, near infrared, or infrared wavelength. Use of multiple wavelengths may allow for cerebral blood oxygenation to be determined. As described below, in some cases, light emitted in an infrared wavelength (e.g., by a laser) may be used to determine cerebral blood volume and/or cerebral blood flow, and light emitted at two different wavelengths (e.g., light emitted in an infrared wavelength and light emitted in a near infrared wavelength) may be used to determined cerebral blood oxygenation. It should be understood that in instances in which two wavelengths of light are used to determined cerebral blood oxygenation, one of the wavelengths may also be used to determine cerebral blood volume and/or cerebral blood flow.

In one particular example, a laser may emit light in a near-infrared wavelength, and the reflected light may be captured by a camera. The reflected light in the near-infrared wavelength range may be used to determine cerebral blood flow and/or cerebral blood volume. Concurrently, an LED may emit light in another near infrared wavelength and the reflected light may be captured by a photodetector. The reflected light from both wavelengths may be used to determined cerebral blood oxygenation. Note that the combination of laser, LED, camera, and photodetector of this example may be considered one “channel,” and a headset may have multiple such channels (e.g., two, four, ten, etc.) disposed at various locations around the headset, each probing a different region of the brain.

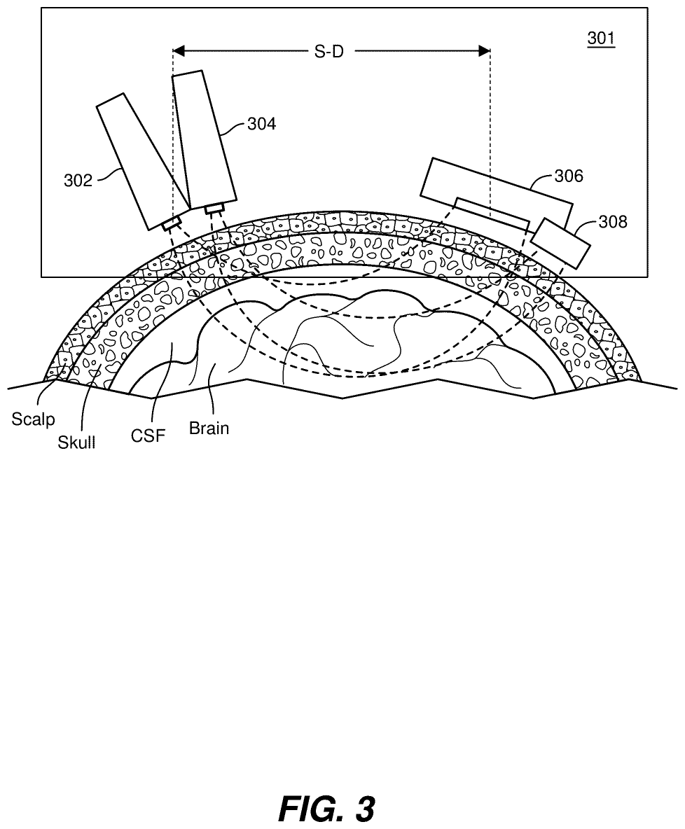

In some embodiments, a light detection package may include multiple light detectors, such as multiple cameras, a camera and a photodetector, or the like. In some implementations, a distance between a light emitter and a corresponding light detector (or between a light emission package comprising two or more light emitters and a corresponding light detection package) may be within a range of about 2.5 cm-4.0 cm. This distance is generally referred to as the “source-detector distance,” and example values may include 2 cm, 2.5 cm, 3 cm, 3.5 cm, 4 cm, etc. In instances in which a headset includes multiple channels (each comprising at least one light emitter or at least one light emission package, and corresponding light detectors or light detection packages), the distance between light emitter and light detector for different channels may be different. In some implementations, positions of light emitters and/or light detectors (or a light emission package and or a light detection package) may be modifiable. For example, a light emission package and/or a light detection package may be affixed to a headband or a headset via screws, Velcro, or other hardware at a position that is modifiable. This may allow for distances between a light source and a light detector to be modified, which may in turn allow the depth of imaging to be modified as different source-detector distances may impact the depth the emitted light can penetrate within the brain, as shown in and described below in connection with FIG. 3. Additionally, modification of positions of light emitters and/or light detectors may allow different brain regions to be probed using one headset.

FIG. 2A illustrates an example apparatus 200 for generating cerebral blood metrics data in accordance with some embodiments. Apparatus 200 includes a headset 201 with a headband 202. As illustrated, headset 201 includes a plurality of channels, each channel including one or more light emitters and one or more light detectors. Each light emitter and each light detector is affixed to a portion of the headband 202 of the headset 201. As illustrated, headband 202 and headset 201 are configured to encircle at least a portion of the head of a user, e.g., at forehead level. The plurality of channels of the headset 201 include a first channel 204.

Referring to FIG. 2B, as illustrated, first channel 204 includes a light emission package 206. Light emission package 206 includes a laser and an LED. As illustrated, first channel 204 also includes two light detectors, 208 and 210, which together may be referred to herein as a “light detection package.” Light detectors 208 and 210 may be cameras, photodiodes, etc.

Note that a headset or headband may include one or more channels (e.g., one, two, four, five, ten, etc.). For example, in some implementations, a headset or headband may include four channels. By way of example, the four channels may be configured to measure cerebral blood metrics on a forehead (or frontal lobe region), a parietal lobe region, or the like. In some embodiments, the channels may be symmetrically disposed with respect to one another. For example, a first channel may be disposed proximate the left parietal lobe, and a second channel may be disposed proximate the right parietal lobe.

FIG. 3 illustrates an example implementation of a channel 301 of a headset in accordance with some embodiments. Elements of channel 301 may be similar or analogous to elements of channel 204 in FIGS. 2A and 2B. As illustrated, channel 204 may include a laser 302 and an LED 304. Laser 302 may be configured to emit light in the infrared wavelength. In one example, laser 302 may emit light at about 830 nm. In another example, laser 302 may emit light at about 785 nm. LED 304 may be configured to emit light at a different wavelength than laser 302. In one example, LED 304 may emit light in the near infrared wavelength. Channel 204 also includes two light detectors, camera 306 and light detector 308 (which may include, e.g., photodiodes). As illustrated, camera 306 may obtain light reflected off various brain structures from light emitted by laser 302. Data captured by camera 306 may be used to determine cerebral blood flow and/or cerebral blood volume metrics, as described below in connection with FIGS. 6 and 7. Light detector 308 may obtain light reflected off various brain structures from light emitted by LED 304. Data captured by camera 306 and light detector 308 may be combined to determine cerebral blood oxygenation metrics, as described below in connection with FIG. 6.

FIG. 4 depicts a schematic drawing of a front view of an example of an apparatus 400 for generating cerebral blood metrics data, according to embodiments. FIG. 5 is a schematic diagram of a top view illustrating components of the apparatus 400 in FIG. 4. Apparatus 400 includes a headset 401 with a headband 402. The headset 401 includes a light emission package 410 with a light source 414 (e.g., laser) and a light detector package 430 with a light detector 436 (e.g. a CMOS board camera). The light emission package 410 and light detector package 430 form a channel 404. The light emission package 410 and light detector package 430 are disposed on a portion of the headband 402. In one implementation, the light emission package 410 and light detector package 430 are adjustable to different positions along the headband 402. As shown, the headband 402 is configured to encircle at least a portion of a head 10 of a patient at, e.g., the forehead level.

The apparatus 400 also includes an electrical connection 438 (e.g., USB cable) between the light detector 436 of the light detector package 430 and one or more processors and/or a computing device for communicating data. In other implementations, apparatus 400 may omit electrical connection 438 and includes a wireless transmitter for communicating data to a wireless receiver in electrical communication with the one or more processors and/or computer readable medium. The processor(s) or computing device may perform operations to determine one or more cerebral blood metrics based on the data captured by the light detector.

In various embodiments, an apparatus for generating cerebral blood metrics data includes a headset configured to be worn on a head of a patient or other user during operation. For example, the headset may include a headband that is a strap, cap or other component that can encircle the head or otherwise attach to the head. The headband may include a tightening mechanism such as Velcro, latch, or the like that can secure and tighten the headband to the head. In some cases, the tightening of the headband may be used to place the light detector(s) in contact with and/or apply pressure to the scalp locally where the light detector(s) contacts the scalp. Contact pressure may be desirable to reduce blood flow at the scalp locally to decrease the scalp's influence and increase brain specificity. In addition, the headband, the one or more light detectors, and/or more or more light sources may be configured to allow adjustment of the locations of the light detector(s) and light source(s) to probe different regions/depths of the brain.

Referring back to FIG. 4, channel 404 includes a light source 414 and a light detector 436 (e.g., a camera such as a board camera). In FIG. 5, a first surface 431 of the detector package 430 may be in contact with the scalp during operation. In some cases, the headband 402 may be tightened to apply a pressure to the scalp at a region where the detector package 430 contacts the scalp to decrease the blood flow in the scalp locally. The light source 414 may be a laser configured to emit coherent laser light in a near-infrared or infrared wavelength into the head 10 of the user. In one example, the laser may emit light at about 830 nm. In another example, the laser may emit light at about 785 nm. The light detector 436 may include one or more CMOS sensors or may be a photodiode. There is a gap between the light emission package 410 and the scalp with a gap distance, D_{gap}. In some cases, the gap distance, D_{gap}, may be in a range of 5 mm and 7 mm. In one example, the gap distance, D_{gap}, may be set to generate an illumination spot with the given light intensity level that lies within safety standards. As illustrated, the light detector 436 may obtain light reflected off various brain structures from light emitted by light source 414 as illustrated by the example light paths 411. Data captured by the light detector 436 may be used to determine cerebral blood metrics data.

Returning to FIG. 5, the illumination spot of the light source 414 and the light detector 436 are at a source-detector distance (S-D distance), D_{sd}, from each other. The S-D distance impacts the depth that the emitted light can penetrate into the brain. In certain implementations, the S-D distance may be adjusted to tune the depth of penetration into the brain. The locations of the light detector 436 and light source 414 determine the region being imaged. In the illustration, the light detector 436 is positioned in contact with the scalp and at the S-D distance from the illumination spot of the light source 414 in order to collect light emerging from the brain at a distance away from the laser illumination spot. In some cases, the sensor area of the light detector 436 may in the range of 25 mm² and 100 mm² and the pixel pitch may be in a range of 2 μm and 4 μm. The spatial distribution of the exiting photons collected by the light detector 436 exhibits a granular pattern in the images with areas of high and low intensity, which are referred to herein as “speckles.” The light detector 436 is typically configured with an exposure time that is significantly larger than the decorrelation time, Tc, of the speckle field. For example, exposure times may be in a range of 1 ms and 10 ms. This results in multiple speckle patterns integrated into a single camera frame. The motions within the light paths 411 are primarily due to movement of blood cells and will scatter and change the effective optical path lengths resulting in a fluctuating speckle field that varies in time. The speckle contrast calculated from the images can be used to determine cerebral blood flow.

In some implementations, the apparatus for generating cerebral blood metric data includes a light source that is a laser (e.g. a laser diode). The laser may be a continuous wave laser. The laser may be configured to emit light in a near-infrared or infrared wavelength. In one example, the laser may emit light at about 830 nm. In another example, the laser may emit light at about 785 nm. In one instance, the light source may be a single-mode continuous wave 785 nm laser diode (e.g., Thorlabs laser L785H1) that can deliver up to 200 mW. In other implementations, other wavelength may be used such as 1064 nm.

In various examples, an apparatus for generating cerebral blood metric data includes a light detector, which may be a camera, or the like. A light detector may be positioned to collect light from the brain and output a data signal including data representative of one or more image frames captured over time. Each image frame may include an intensity distribution of light received at a sensing region. The light detector may be configured to operate at a frame rate (e.g., 50 frames per second (fps)) and with a pixel pitch (e.g., 3.4 μm). The light detector may include one or more sensors. Some examples of suitable sensors include a complementary metal-oxide-semiconductor (CMOS) sensor, a linear or array charge-coupled device (CCD), and other similar devices. In one embodiment, the sensor may be a CMOS sensor in a flexible format such as the Cappella CMOS image sensor sold by Teledyne. In one embodiment, the light detector is a USB-Board camera (e.g., the Basler daA1920-160 μm camera with a Sony Sensor IMX392). In some cases, the light detector may include one or more CMOS sensors and have a width in a range of 5 mm and 10 mm and a length in a range of 5 mm and 10 mm. In some cases, the pixels of a CMOS sensor may have a size in a range of 2 μm and 4 μm.

According to one aspect, a light detector may include multiple segments. This segmentation allows data from multiple source-detector distances to be measured simultaneously with a single light detector. For example, the light detector may include multiple sensors such as multiple CMOS sensors. The CMOS sensors may be arranged at different S-D distances from the light source such that the frames are captured of regions at different depths. As another example, the light detector may include one sensor with different sets (segments) of pixels at different locations.

In some implementations, cerebral blood metrics may include one or more of cerebral blood flow, cerebral blood volume, and/or cerebral blood oxygenation. In some embodiments, cerebral blood volume may be determined based on the intensity of reflected light as measured by a light detector. Note that cerebral blood volume may be determined based on reflected light of a single wavelength (e.g., emitted from a laser, from an LED, etc.). In some implementations, cerebral blood oxygenation may be determined based on the intensity of reflected light at two wavelengths (e.g., emitted by a laser and an LED), and determining oxygenation based on the differential optical transmission changes for the two different wavelengths. For example, the two wavelengths may be one where oxyhemoglobin absorbs more light and one where deoxyhemoglobin absorbs more light. The two wavelengths may be in the visible, near-infrared, or infrared wavelength range. In some embodiments, known formulas for calculating oxygen saturation (e.g., based on the Beer-Lambert Law, or other similar techniques) may be utilized to determine cerebral blood oxygenation metrics using two wavelengths.

In some embodiments, cerebral blood flow may be determined using collected scattered light at a single wavelength (e.g., light emitted in an infrared wavelength range). Note that wavelength choice may affect penetration depth of the light. For example, light emitted in the infrared wavelength range may penetrate to deeper structures relative to near infrared light. In some implementations, cerebral blood flow may be based on diffuse correlation spectroscopy (DCS), or on speckle visibility spectroscopy (SVS), which is also referred to as speckle contrast optical spectroscopy (SCOS). In general, a “speckle” refers to the pattern of bright and dark spots in an image resulting from scattering of illuminated laser light (e.g., scattered by the scalp, skull, and/or brain) resulting from constructive and destructive interference of the light. The speckle pattern dynamics change with blood flow dynamics. The time that it takes one speckle pattern to change to a different speckle pattern is referred to as the decorrelation time, which is correlated with cerebral blood flow rate. Both DCS and SCOS are techniques for measuring how fast the speckle pattern changes, and hence, estimating cerebral blood flow rate. DCS may determine changes in speckle decorrelation time based on a single pixel of the speckle image, i.e., requiring a fast photodetector to detect the light. In contrast, SCOS may determine speckle decorrelation time using a relatively slow camera with a large number of pixels. In particular, the camera exposure time, typically in the range of 0.3 milliseconds to 15 milliseconds, may be set to be substantially longer than the decorrelation time of the speckle field (typically a few tens of microseconds), which may result in multiple different speckle patterns summed up into a single camera frame. As the speckle field fluctuates, the speckle pattern recorded by the camera is smeared and washed out within the exposure time. Accordingly, the cerebral blood flow may be quantified from the degree of blurring of the captured frame, which is generally referred to herein as the speckle contrast. Note that use of SCOS to measure cerebral blood flow may have the advantage that a relatively inexpensive camera may be used as a light detector, because a high frame rate is not needed, unlike detectors used for DCS. Moreover, the camera may be directly mounted on a headset or headband, which may eliminate optical loss associated with a light guide running from the head to the camera, which may introduce its own noise and motion artifacts. In some examples, a camera used for SCOS may have an integration time of within a range of about 0.3 milliseconds-15 milliseconds. The camera may use a frame rate of within a range of about 30 frames per second-150 frames per second. In one example, the camera may have a frame rate of about 80 frames per second.

Speckle contrast may be determined at each camera frame as:

$$K = \frac{{σ}_{I}}{\overline{I}} = \frac{\sqrt{〈 {I}_{j}^{2} 〉 - {〈 {I}_{j} 〉}^{2}}}{〈 {I}_{j} 〉} \qquad ( Eqn . 1 )$$

In the equation given above, I_{J} represents the instantaneous intensity recorded at the camera at pixel J, σ_{I} represents the standard deviation of I, and Ī represents the mean of I. The speckle decorrelation time is determined as:

$$rCBF ∝ \frac{1}{τ} = \frac{1}{β {K}^{2}} \qquad ( Eqn . 2 )$$

The speckle decorrelation time is directly correlated with the cerebral blood flow rate. The relative cerebral blood flow, measured in units of blood flow index (BFI), is inversely correlated with τ and therefore inversely correlated with βK². The correction factor β is a constant depending on system setup, e.g. speckle size, pixel size, and polarization of the laser light.

FIG. 6 illustrates use of DCS and SCOS for determining cerebral blood flow in accordance with some embodiments. As illustrated, DCS samples a single fluctuating speckle at a relatively high rate, and calculates the cerebral blood flow from the time trace of the intensity at a single point. In particular, graph 602 illustrates the fluctuating intensity as a function of time of a speckle at a single point, and graph 604 illustrates the cerebral blood flow rate determined based on an auto correlation of the fluctuating intensity depicted in graph 602. SCOS captures an integrated speckle pattern over time, where the speckle fluctuates more quickly over time at higher cerebral blood flow rates, leading to washout in the captured image. By measuring the extent of the washout or blurring in the captured image (e.g., the speckle contrast), cerebral blood flow can be determined. Images 606 illustrate the speckle patterns that are integrated in a single image to determine speckle contrast, which is used to determine cerebral blood flow as a function of time, as illustrated in graph 608.

FIG. 7 illustrates example graphs of cerebral blood volume, cerebral blood flow, and cerebral blood oxygenation as a function of time in accordance with some embodiments. As described above, these cerebral blood metrics may be obtained over a period of time, where, during a subset of the period of time, the patient is holding their breath. Breath holding time period 702 is illustrated in FIG. 7. Note that, cerebral blood metrics may be obtained over a baseline time period, generally referring to the time period during which cerebral blood metrics are obtained prior to initiation of breath holding. Baseline time period 704 is illustrated in FIG. 7. Cerebral blood metrics may also be obtained during a recovery time period 706, which generally begins at a time from when breath holding ends (e.g., the end of breath holding time period 702).

Note that each time period may be any suitable duration of time. Example duration of breath holding may be 15 seconds, 30 seconds, 45 seconds, 60 seconds, etc. In some embodiments, the duration of breath holding may be however a long a patient can hold their breath relatively comfortably (e.g., the breath holding time period may vary for different people). In some implementations, the baseline time period and/or the recovery time period may be at least as long as the breath holding time period. In some embodiments, the recovery time period may be longer than the breath holding time period. In some embodiments, the recovery time period may be a time duration long enough that cerebral blood flow metrics return to within a predetermined range of the corresponding values during the baseline time period. In some such embodiments, the recovery time period may be dynamically adjusted. For example, the recovery time period may be stopped responsive to determined that cerebral blood metrics have returned to baseline values.

FIG. 8 illustrates examples of experimental cerebral blood metric data collected during a breath holding task in accordance with some embodiments. As illustrated by graph 802, cerebral blood flow is determined during a time period that includes the patient holding their breath. Similarly, as illustrated by graph 804, cerebral blood volume is determined over the same time period. Note that cerebral blood flow and cerebral blood volume may be determined using light emitted at a single wavelength (e.g., in the infrared wavelength region) and the captured reflected light data at the single wavelength. As described above, cerebral blood flow may be determined based on speckle decorrelation time (e.g., using DCS or SCOS, as described above), and cerebral blood volume may be determined based on the intensity of the reflected light. For example, cerebral blood volume may be determined as the ratio of the mean time-varying intensity of the reflected light to the baseline intensity. The blood volume was extracted from the camera images by calculating the ratio of the mean time-varying intensity over the baseline intensity (calculated by taking the mean image intensity of the first 60 seconds or less). It is calculated as follow:

$$CBV = \frac{2 {I}_{0} - 〈 I ( t ) 〉}{{I}_{0}}$$

or can also be calculated as CBV=log₁₀ (I₀/<I(t)>). In the example data shown in FIG. 8, cerebral blood flow is determined using SCOS. Note that graphs 802 and 804 indicate a baseline time period 806 prior to initiation of breath holding, a breath holding time period 808 during which time the patient is holding their breath, and a recovery time period 810 after the patient resumes normal breathing.

As illustrated in graphs 802 and 804, both cerebral blood flow and cerebral blood volume increase during breath holding time period 808. This is attributed to the brain's increased demand for blood to transport oxygen and carbon dioxide until breath holding stops. During the breath holding time period 808, the brain enters a heightened state of alert which triggers a sequence of protective mechanisms to ensure stable regulation of carbon dioxide and oxygen, which is achieved through accelerated circulation of blood leading to increased blood flow together with an increase in blood volume in the brain via dilation of blood vessels.

Additionally, note that both cerebral blood flow rates and cerebral blood volume remain elevated at the beginning of recovery time period 810 prior to recovery to the baseline level of each metric.

Plots 812 and 814 illustrate subsets of cerebral blood flow graph 802 over short time scales. Similarly, plots 816 and 818 illustrate subsets of cerebral blood volume graph 804 over short time scales. Note that the pulsations evident in each of plots 812, 814, 816, and 818 are not noise but represent blood pulsations. As illustrated in frequency domain graph 820, the pulsations may be used to determine a heart rate of the patient (e.g., based on the periodicity of the pulsations). For example, heart rate may be determined by taking a Fourier transform of time domain data. Additionally, note that cerebral blood flow and cerebral blood volume may capture different details in blood flow. For example, the dicrotic notch and peak pressure are discernible in plot 814 of the cerebral blood flow.

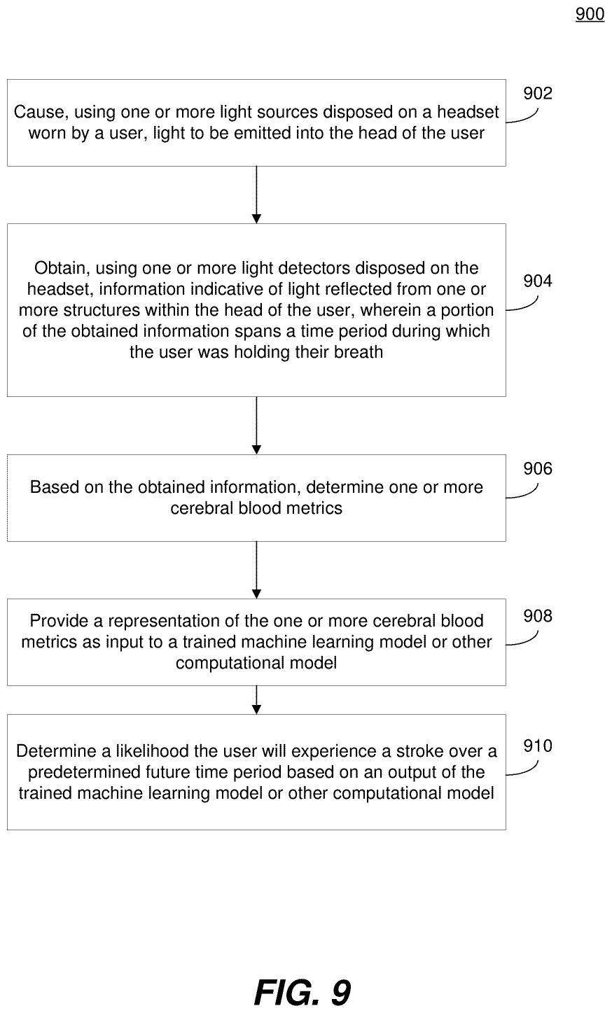

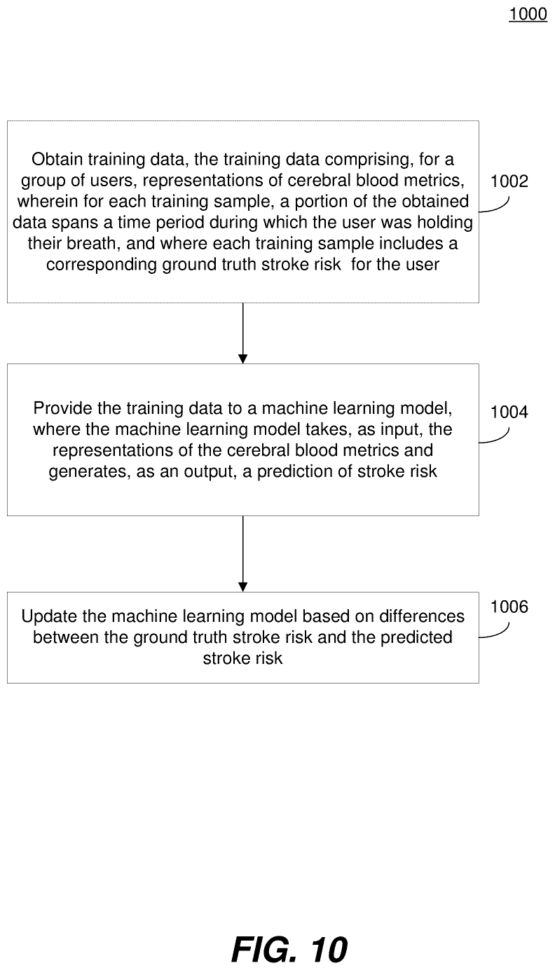

In some implementations, raw data traces of cerebral blood metrics (e.g., cerebral blood volume, cerebral blood flow, and/or cerebral blood oxygenation as a function of time) may be provided to a trained machine learning model configured to output a stroke likelihood prediction. Such a machine learning model that accepts raw data traces may be a DNN or other suitable architecture. Alternatively, in some embodiments, one or more features associated with the cerebral blood metrics may be extracted. The extracted features may then be provided to a trained machine learning model, which in turn may generate a predicted stroke likelihood. The extracted features may be considered “a representation of the one or more cerebral blood flow metrics.” In instances in which one or more extracted features are provided to a machine learning model, the model may be a perceptron, a random forest, or any other suitable architecture. Note that techniques for generating predicted stroke likelihoods using a machine learning model are shown in and described below in connection with FIG. 9, and techniques for training such a model are shown in and described below in connection with FIG. 10.

In some implementations, extracted features may include information obtained during a time a patient or other user was able to hold their breath (generally referred to herein as T_{BH}) or during administering of a gas mixture having a predefined concentration of CO₂.

In some implementations, extracted features may include percentage change in a cerebral blood metric at a maximum or minimum after initiation of breath holding compared to a baseline value. For example, referring to FIG. 7, the percentage change in cerebral blood volume (CBV change), the percentage change in cerebral blood flow (CBF change), and the percentage change in cerebral blood oxygenation (CBO change) relative to the baseline value) are indicated. Note that to determine a percentage change, each cerebral blood metric may be normalized based on the baseline value.

In some implementations, the extracted features may include a rate of change (e.g., a slope) in the change in a cerebral blood metric during breath holding. For example, rate of change of cerebral blood flow may be determined by dividing a percentage change of cerebral blood flow (e.g., as indicated in FIG. 7) by the duration of time the patient holds their breath, to derive a feature with units of percent change per second. Similarly, an extracted feature may include a rate of recovery (e.g., a slope) in the change in a cerebral blood metric after resuming normal breathing. Note that, in some implementations, rates of change either during the breath holding time period, or a rate of change associated with recovery, may be determined by fitting a function (e.g., an exponential function) to a portion of the cerebral blood metric, and estimating rate of change metrics based on a growth or decay constants from the fitted function.

In some implementations, the extracted features may include a ratio of a percentage change of one cerebral blood metric to a percentage change of another cerebral blood metric. In one example, an extracted feature may be a ratio of the percentage change of cerebral blood flow (e.g., CBF change in FIG. 7) to the percentage change of cerebral blood volume (e.g., CBV change in FIG. 7). Other examples include a ratio of the percentage change of cerebral blood flow to cerebral blood oxygenation, and/or a percentage change of cerebral blood volume to cerebral blood oxygenation. In some implementations, the extracted features may include a ratio of rate of change of one cerebral blood metric to a rate of change of another cerebral blood metric. Examples include a rate of change of cerebral blood flow to a rate of change of cerebral blood volume, a rate of change of cerebral blood flow to a rate of change of cerebral blood oxygenation, or a rate of change of a cerebral blood volume to a rate of change of cerebral blood oxygenation. Note that ratios of rates of change may be determined based on rate of change either during the breath holding time period, or during a recovery time period after normal breathing resumes.

In some implementations, extracted features may include fine grained features from within a cardiac cycle as observed within a cerebral blood flow trace. For example, referring to FIG. 8, graph 814 illustrates three peaks, labeled P1, P2, and P3, within the cerebral blood flow trace in graph 814. Note that all of P1, P2, and P3 are from within a single cardiac cycle, where the cerebral blood flow trace includes multiple (e.g., hundreds) of cardiac cycles, each with their own peaks. In general, P1 corresponds to the rapid ejection of blood during systole, the second peak P2 corresponds to reflected waves from the vascular tree, and P3 corresponds to the dicrotic notch at the beginning of diastole. In some embodiments, ratios of the amplitude of any of these peaks (P1 to P2, P2 to P3, and/or P1 to P3) may be used to form an extracted feature. The ratio may be taken from a cardiac cycle extracted from the baseline time period, during the breath holding time period, or during the recovery time period. In some implementations, a ratio of a peak ratio from the breath holding time to a peak ratio from a baseline or recovery time period may be considered an extracted feature. By way of example, the ratio of the P2 peak to the P1 peak during breath holding may be determined and represented as the breath holding peak ratio. Continuing with this example, the ratio of the P2 peak to the P1 peak during the baseline time period may be determined and represented as the baseline peak ratio. An extracted feature may then be the breath holding peak ratio to the baseline peak ratio.

Note that extracted features may be extracted and/or determined autonomously (e.g., without user input) upon collection of the one or more cerebral blood flow metrics. The extracted features may then be provided to a trained machine learning model configured to generate the prediction of stroke likelihood for the patient. Note that any suitable number or combination of extracted features may be utilized.

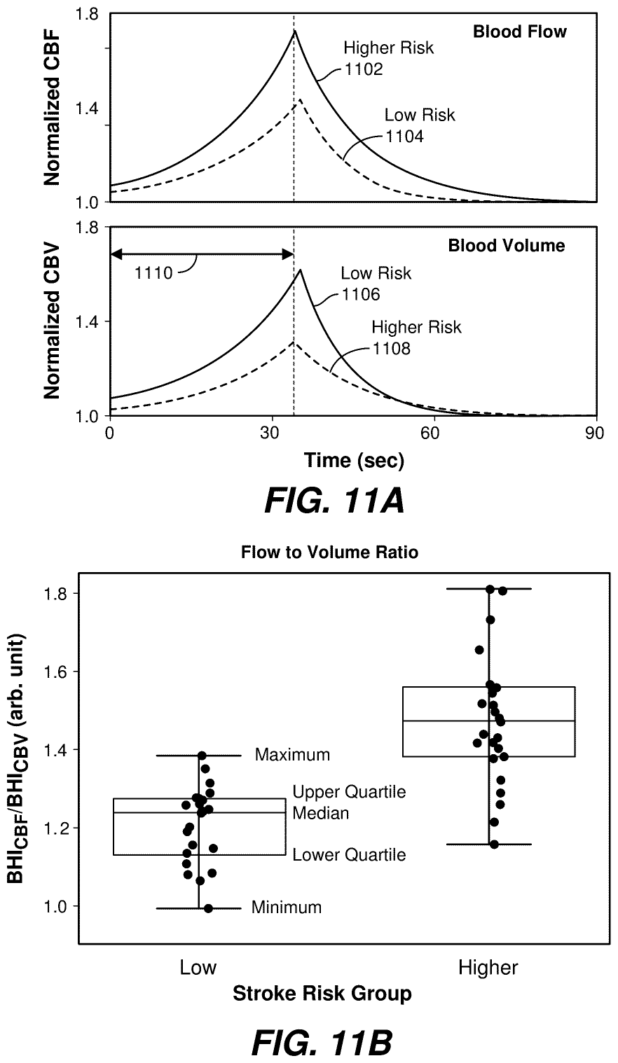

Alternatively, in some embodiments, rather than utilizing a trained machine learning model, stroke risk may be predicted based on extracted features, e.g., by comparing an extracted feature to one or more predetermined thresholds. By way of example, stroke risk may be classified as “high risk” or given a high risk score (e.g., 7 on a scale of 1-7, 10 on a scale of 1-10, 1.0 on a scale of 0-1.0, etc.) responsive to determining that a value of a particular extracted feature (e.g., the percentage change in cerebral blood flow) exceeds a predetermined threshold. As another example, stroke risk may be classified as “high” or a high risk score responsive to determining that a value of a particular extracted feature (e.g., the percentage change in cerebral blood volume) is below a predetermined threshold. In one example, stroke likelihood may be determined based on a ratio of a percentage change in cerebral blood flow (between a peak cerebral blood flow value after the user begins holding their breath and a baseline cerebral blood flow to a percentage change in cerebral blood volume (between a peak cerebral blood volume after the user begins holding their breath and a baseline cerebral blood volume), as shown in and described below in connection with FIGS. 11A and 11B. In particular, the ratio may be determined, and in some cases, compared to a predetermined threshold to generate a stroke likelihood. In some cases, multiple extracted features may be considered and may be compared to predetermined thresholds, and a stroke risk may be determined based on an aggregation of the multiple extracted features. For example, a stroke risk may be categorized as “high” or a high risk score responsive to more than 50%, 60%, 70%, etc. of extracted features meeting threshold criteria for high stroke risk. Note that, in such instances, a computing device may store thresholds for categorizing stroke risk based on any suitable extracted features, and may perform such comparisons to generate a stroke risk. Such a computing device may be disposed on a headset, or may be communicatively coupled to a processor or other computing device disposed on the headset.

In one example, extracted features may be based on a breath holding index (BHI), generally defined herein as the maximal change from baseline during breath-holding divided by the duration of breath-holding T_{BH} for a given cerebral blood metric. For example, BHI for cerebral blood flow may be defined by:

$${BHI}_{CBF} = 100 * \frac{{CBFI}_{max} - {CBFI}_{0}}{{CBFI}_{0} * {T}_{BH}} \qquad ( Eqn . 3 )$$

Similarly, the BHI for cerebral blood volume may be defined by:

$${BHI}_{CBV} = 100 * \frac{{CBVI}_{max} - {CBVI}_{0}}{{CBVI}_{0} * {T}_{BH}} \qquad ( Eqn . 4 )$$

In some examples, the flow to volume ratio may be an extracted feature used to predict stroke risk. The flow to volume ratio may be determined based on the ratio of the BHI for cerebral blood flow to the BHI for cerebral blood volume:

$$Flow to volume ratio : \frac{{BHI}_{CBF}}{{BHI}_{CBV}} \qquad ( Eqn . 5 )$$

In some cases, BHI_{CBF} may have a value within a range of about 0% to 5%, and BHI_{CBV} may have a value within a range of about 0% to 3%. The flow to volume ratio may have a value within a range of about 0.9 to 2. A threshold for categorizing a patient as having relatively high stroke risk may be based on the flow to volume ratio (e.g., the patient may be categorized as high risk if the flow to volume ratio is greater than 1.2, greater than 1.3, greater than 1.4, etc.) FIG. 11B presents experimental data depicting the flow to volume ratio using the BHI for patients deemed to be high risk or low risk based on stroke questionnaire data.

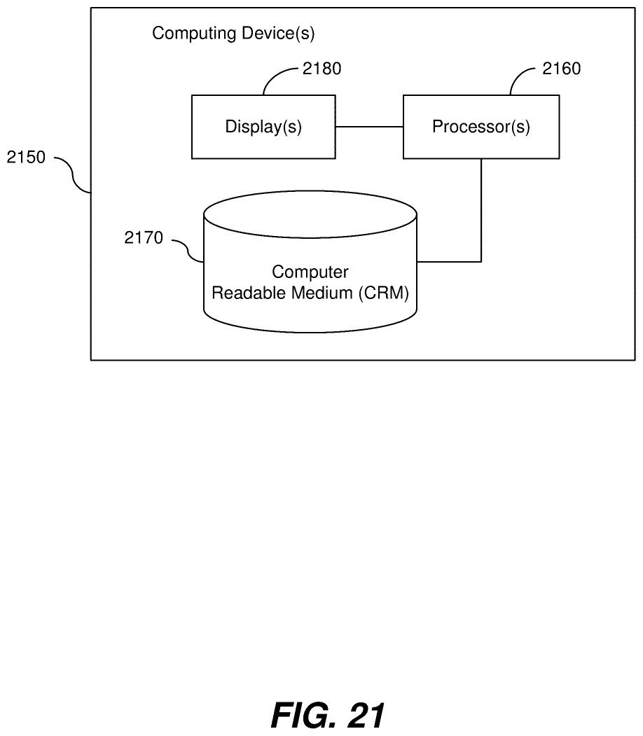

FIG. 9 is a flowchart of an example process 900 for determining a stroke likelihood using cerebral blood metrics in accordance with some embodiments. Blocks of process 900 may be executed by one or more processors of one or more computing devices. An example of such a computing device is shown in and described below in connection with FIG. 21. Note that, in some embodiments, at least one of the one or more computing devices may be disposed on a headband or headset on which the one or more light sources and light detectors are disposed. Accordingly, in some such embodiments, cerebral blood metrics, extracted features associated with the cerebral blood metrics, and/or the stroke likelihood may be determined by a computing device itself on the headband or headset. Alternatively, in some embodiments, data obtained by the one or more light detectors, and/or data representative of the cerebral blood metrics may be transmitted from a computing device disposed on the headband or headset to a second computing device remote from or separate from the headband or headset, where the second computing device generates the stroke likelihood. In some implementations, blocks of process 900 may be executed in an order other than what is shown in FIG. 9. In some embodiments, one or more blocks of process 900 may be omitted, and/or two or more blocks may be executed substantially in parallel.

Process 900 can begin at 902 by causing, using one or more light sources disposed on a headset worn by a user, light to be emitted into the head of the user. An example of such a headset is shown in and described above in connection with FIGS. 2A and 2B. Another example of such a headset is shown in and described above in connection with FIGS. 4 and 5. The one or more light sources may include one or more lasers, one or more LEDs, etc. As described above, in some implementations, two light sources of different types (e.g., a laser and an LED), each of which may emit light in a different wavelength region (e.g., infrared and near infrared) may be packaged together as a light emission package. In some implementations, multiple light emission packages may be disposed on a headset or headband, each configured to emit light into different regions of the user's head or brain. Note that light may be emitted continuously, or may be pulsed.

At 904, process 900 may obtain, using one or more light detectors disposed on the headset, information indicative of light reflected from one or more structures within the head or brain of the user, wherein a portion of the obtained information spans a time period during which the user was holding their breath. An example of one or more light detectors disposed on a headband of a headset is shown in and described above in connection with FIGS. 2A and 2B. An example of one or more light detectors disposed on a headband of a headset is shown in and described above in connection with FIGS. 4 and 5. As described above, the one or more light detectors may include a camera, a photodetector, etc. In instances in which a light emission package includes two light sources each emitting light in a different wavelength range, a corresponding light detection package may include two light detectors, each configured to receive reflected light corresponding to emissions from the corresponding light emitter. In one example, light emitted by a laser may be reflected from various head and brain structures and may be captured by a camera (e.g., to determine speckle contrast, as described above), and light emitted by an LED may be reflected and captured by a photodetector. Note that, as shown in and described above in connection with FIGS. 2A and 2B, because a headset may include multiple (e.g., two, four, eight, ten, etc.) light emission packages and corresponding light detection packages, the obtained information may correspond to different brain regions (e.g., a left frontal lobe region, a right frontal lobe region, a left parietal lobe region, a right parietal lobe region, etc.).

Note that, as described above in connection with FIGS. 7 and 8, the obtained light reflection data spans a baseline time period, a breath holding time period, and a recovery time period. In some implementations, process 900 may cue the user to begin holding their breath at a particular time. For example, the cue may be an audible cue (e.g., a spoken instruction, an audible beep, etc.), or may be a haptic cue (e.g., a vibration delivered using the headband or other components of a headset).

At 906, process 900 can, based on the obtained information, determine one or more cerebral blood metrics. As described above, the cerebral blood metrics may include cerebral blood volume, cerebral blood flow, and/or cerebral blood oxygenation. Process 900 may additionally determine a duration of time the user held their breath. The duration of time may be determined by, e.g., thresholding any of the cerebral blood metrics to determine a time point at which the cerebral blood metric began deviating from baseline to the time point at which the cerebral blood metric reached a minimum or maximum value. Note that, as described above, cerebral blood volume may be determined based on intensity of the reflected light signal at a single wavelength. Cerebral blood flow may be determined using DCS and/or SCOS (as shown in and described above in connection with FIG. 6). Cerebral blood oxygenation may be determined based on the ratio of reflected light at two different wavelengths (e.g., in instances in which light is emitted by at least two light sources at two different wavelengths, such as an infrared wavelength and a near infrared wavelength).

At 908, process 900 can provide a representation of the one or more cerebral blood metrics as input to a trained machine learning model or other computational model. Note that the representation of the one or more cerebral blood metrics may include the raw data of the cerebral blood metrics as a function of time, or, alternatively, may include one or more extracted features extracted from the one or more cerebral blood metrics. Examples of extracted features are described above.

At 910, process 900 can determine a likelihood the user will experience a stroke over a predetermined future time period based on an output of the trained machine learning model or other computational model. For example, the likelihood may correspond to a time period of the next year, the next five years, the next ten years, the remainder of their lifetime, etc. The likelihood may be provided as a discrete classification (e.g., low risk, medium risk, high risk), a continuous value (e.g., a continuous probability value), or in any other suitable format.

In some implementations, the stroke likelihood may be presented in any suitable manner. For example, the stroke likelihood may be audibly presented (e.g., by speakers associated with the headset, or by speakers of an associated or paired device). The stroke likelihood may be visibly presented, e.g., on a display of an associated or paired device. In some implementations, the stroke likelihood may be automatically (e.g., without user input) stored in an electronic medical record associated with a patient, e.g., such that a physician can review the stroke likelihood. In cases in which the stroke likelihood is stored, e.g., as medical data, the stroke likelihood may be stored in conjunction with timestamp information indicating a date and/or time the stroke likelihood prediction was made. This may allow a physician or other healthcare provider to monitor changes in the stroke likelihood for a given patient over time. This may allow the healthcare provider to determine whether various interventions are modifying stroke likelihood (e.g., lowering the risk of stroke) over time. In some implementations, an updated stroke likelihood may be determined after a first stroke likelihood is determined (e.g., two months later, six months later, a year later, two years later, etc.) A difference between the first stroke likelihood and the updated stroke likelihood may be determined, e.g., to determine if the patient's likelihood of experiencing stroke remains the same over time, is increasing over time, or is decreasing over time. In some embodiments, a recommendation may be generated based on the change in stroke likelihood over time. For example, a recommendation to continue or implement particular lifestyle modifications may be made, a recommendation to initiate a particular medical treatment may be made, etc. In some embodiments, an indication of the change in stroke likelihood over time may be transmitted to a user device associated with the patient, with a healthcare provider, etc.

A machine learning model may be trained using a training set that includes representations of cerebral blood metrics (which may include cerebral blood flow, cerebral blood volume, and/or cerebral blood oxygenation as a function of time, or extracted features as discussed above), and corresponding ground truth data. The training set may include data associated with multiple patients, who may be of varying demographics (e.g., different lifestyles, different ages, different sexes, etc.). In some embodiments, ground truth data may be questionnaire data, where the questionnaire predicts a stroke likelihood based on demographic data, lifestyle data, physiological metrics (e.g., blood pressure, resting heart rate, etc.), and the like. Alternatively, in some embodiments, ground truth data may be actual stroke occurrence data, e.g., obtained from longitudinal data that collects representations of cerebral blood metrics from a set of patients who are followed over time. Continuing with this example, the ground truth data may indicate that the patient did not have a stroke if no stroke was recorded during the duration of time the longitudinal study occurred, or, conversely, may indicate that the patient did have a stroke if such stroke occurred. Regardless of how ground truth data is obtained, a machine learning model may be trained by providing representations of cerebral blood flow metrics as input, obtaining a stroke risk prediction based on the input, and updating weights of the model based on a difference between the predicted stroke risk and the ground truth stroke risk. This procedure may be implemented to train a DNN that operates on traces of cerebral blood metrics as a function of time, and/or a perceptron, random forest, or other type of network that operates on extracted features associated with cerebral blood metrics or associated with aspects of the breath holding task (such as duration of breath holding).

FIG. 10 is a flowchart of an example process 1000 for training a machine learning model in accordance with some embodiments. Blocks of process 1000 may be executed by one or more computing devices, such as a server device, a desktop computer, a laptop computer, etc. Note that the one or more computing devices may be different than the one or more computing devices that execute blocks of process 900. In some implementations, blocks of process 1000 may be executed in an order other than what is shown in FIG. 10. In some embodiments, two or more blocks of process 1000 may be executed substantially in parallel. In some embodiments, one or more blocks of process 1000 may be omitted.

Process 1000 can begin at 1002 by obtaining training data, the training data comprising, for a group of users, representations of cerebral blood metrics, wherein for each training sample, a portion of the obtained data spans a time period during which the user was holding their breath, and where each training sample includes a corresponding ground truth stroke risk for the user. As described above, the representations of cerebral blood metrics may include one or more cerebral blood metrics (e.g., cerebral blood flow, cerebral blood volume, and/or cerebral blood oxygenation) as a function of time, and/or extracted features associated with the cerebral blood metrics and/or the breath holding task. Note that cerebral blood metrics for the training data may have been collected using one or more light sources and/or one or more light detectors disposed on a headband similar to the one shown in and described above in connection with FIGS. 2A and 2B. The cerebral blood metrics may be determined based on collected light reflectance data as described above. Ground truth stroke risk data may be questionnaire based, or may be actual stroke occurrence data based on a longitudinal following of the users over time.

At 1004, process 1000 can provide the training data to a machine learning model, where the machine learning model takes, as input, the representations of the cerebral blood metrics and generates, as an output, a prediction of stroke risk.

At 1006, process 1000 can update the machine learning model based on differences between the ground truth stroke risk and the predicted stroke risk. For example, weights of the model may be updated based on a loss function that considers the difference between the ground truth stroke risk and the predicted stroke risk. Note that model updating may be performed for each training sample, or for a batch of training samples.

Note that data (e.g., cerebral blood metric data) included in the training set may be obtained using a headset or other head-worn device, and the trained machine learning model resulting from such a training set may be used to generate stroke risk predictions based on cerebral blood metrics obtained using the same headset or head-worn device, or a different but similar headset or head-worn device. For example, a similar device may be one in which light emitted by one or more light emitters is at about the same wavelength, where source-detector distance is about the same, or the like.

FIGS. 11A and 11B illustrate experimental data in accordance with some embodiments. To obtain the experimental data depicted in FIGS. 11A and 11B, cerebral blood flow (labeled CBF) and cerebral blood volume (labeled CBV) was obtained for fifty (50) patients. The patients were divided into a “low risk” group and a “higher risk” group, determined based on stroke questionnaire data. CBF and CBV were determined using the light emittance and light reflectance techniques described above, in particular, using a laser emitting infrared light, and a camera configured to obtain reflected light. Cerebral blood flow was determined using SCOS, and cerebral blood volume was determined based on intensity of captured reflected light.

As illustrated in FIG. 11A, curve 1102 depicts the cerebral blood flow for patients deemed high risk, and curve 1104 depicts the cerebral blood flow for patients deemed low risk. The breath holding time period 1110 is marked. Note that curves 1102 and 1104 are generated by normalizing and averaging cerebral blood flow across all of the patients in a given risk group. Note that the maximal cerebral blood flow is higher for the patients deemed high risk (represented in the higher peak in cerebral blood flow for the high-risk group) compared to the low-risk group. This is presumed to be due to the less flexible blood vessels of the high-risk group, which causes an impedance in blood vessel dilation during breath holding, which triggers accelerated blood flow.

Conversely, as shown by curves 1106 and 1108, the cerebral blood volume for the low-risk group peaks at a higher level than for the high-risk group. The difference in peaks in cerebral blood flow and cerebral blood volume (i.e., the percentage change in cerebral blood flow from baseline and the percentage change in cerebral blood volume from baseline) were found to be significant across the low risk and high-risk groups, and accordingly, may be strong predictors of stroke risk. Additionally, note that because the peak change is in opposite directions for cerebral blood flow versus cerebral blood volume (in other words, the low risk group had a higher cerebral blood volume and a lower cerebral blood flow, and vice versa for the high risk group), the ratio of the percentage change of cerebral blood flow to percentage change of cerebral blood volume was found to be a highly significant predictor of stroke risk.

FIG. 11B illustrates box plots of the ratio of the percentage change of cerebral blood flow to the percentage change of cerebral blood volume for low-risk and high-risk groups in box plot form. For the data shown, percentage change is measured from the peak after initiation of breath holding to a baseline value using the BHI, as described above, for both cerebral blood flow and cerebral blood volume. The ratio is determined as the BHI of cerebral blood flow to the BHI of cerebral blood volume. The data illustrated in FIG. 11B shows a statistically significant difference between patients assessed to be at low risk of stroke and those assessed to be at high risk of stroke, validating that the ratio of percentage change of cerebral blood flow to the percentage change of cerebral blood volume is a robust predictor of stroke risk.

### Examples of Physiological Metric(s) Data

In various examples disclosed herein, systems for determining stroke risk include stroke prediction engines. For example, the system for determining stroke risk 100 in FIG. 1 includes stroke prediction engine 102. As another example, the system for determining stroke risk 1300 in FIG. 13 includes a stroke prediction engine 1302. Stroke prediction engines may include computational models (e.g., a machine learning model) configured to take, as input, raw physiological metric(s) data (e.g., raw cerebral perfusion metrics data) or one or more extracted features of the raw physiological metric(s) data, and generate, as output, stroke likelihood data. For example, raw physiological metric(s) data such as time traces of cerebral perfusion metrics data (e.g., cerebral blood flow (CBF), cerebral blood volume (CBV) and cerebral blood oxygenation (CBO)) may be used as input into a computational model. Examples of time traces of cerebral blood flow (CBF), cerebral blood volume (CBV) and cerebral blood oxygenation (CBO) are illustrated in the plots in and described in connection with FIG. 7. As another example, one or more features extracted from physiological metrics data may be used as input into a computational model. Some examples of other types of computational models that may be used include machine learning, deep neural networks, Bayesian networks, support vector machines or other kernel-based methods capable of handling physiological inputs. Hybrid models that combine physiological modeling (e.g. extracted features) with data-driven learning approaches such as machine learning or deep neural networks may also be used.

In some implementations, extracted features may be based on physiological metrics data derived from information obtained during a time period including time that a user was able to hold their breath (generally referred to herein as breath-holding time period or T_{BH}) or time that the user is administered a gas mixture having a predefined concentration of CO₂ (generally referred to herein as administering gas mixture time period or T_{AG}).

Some examples of features that can be extracted include: duration of breath holding (τ_{BH}), duration of administering gas mixture with predefined concentration of CO₂ (τ_{AG}), growth factor of cerebral blood flow (τ_{growth}), decay factor of cerebral blood flow (τ_{decay}), percentage change in cerebral blood flow index (CBFI_{change}), percentage change in cerebral blood volume index (CBVI_{change}), breath-holding index for cerebral blood flow (BHI_{CBF}), administering gas mixture index for cerebral blood flow (AGI_{CBF}), breath-holding index for cerebral blood volume (BHI_{CBV}), administering gas mixture index for cerebral blood volume (AGI_{CBV}), ratio of changes from baseline of cerebral blood flow over blood flow volume during breath holding period (BHI_{CBF}/BHI_{CBV}), ratio of changes from baseline of cerebral blood flow over blood flow volume during administering gas mixture time period (AGI_{CBF}/AGI_{CBV}), resting heart rate, maximum heart rate, peak ratio of cardiac pulse during resting period (peaks ratio_{resting}), peak ratio of cardiac pulse during breath-holding period (peaks ratio_{breath-holding}), peak ratio of cardiac pulse during administering gas mixture time period (peaks ratio_{administering gas}), ratio of peaks ratio_{resting} to peaks ratio_{breath-holding} (peaks ratio_{resting}/peaks ratio_{breath-holding}), ratio of peaks ratio_{resting} to peaks ratio_{administering gas} (peaks ratio_{resting}/peaks ratio_{administering gas}),

$${τ}_{mCBVI}^{max} - {τ}_{mCBFI}^{max} ,$$

and other features.

$${τ}_{mCBVI}^{max}$$

refers to the time after breath-holding period or administering gas mixture period at which cerebral blood volume is maximal.

$${τ}_{mCBFI}^{max}$$

refers to time after breath-holding period or administering gas mixture period at which cerebral blood flow is maximal. BHI_{CBF}/BHI_{CBV} and AGI_{CBF}/AGI_{CBV} are sometimes referred herein to as flow-to-volume change features. In some cases, the physiological metric(s) data may be cerebral blood metric data including, for example, cerebral blood flow (CBF), cerebral blood volume (CBV) and/or cerebral blood oxygenation (CBO).

In one example, extracted features may be based on an administering gas mixture index (AGI), generally defined herein as the maximal change from baseline during administering a gas mixture with a predefined CO₂ concentration divided by the duration of the administering time period T_{AG} for a given cerebral blood metric. For example, AGI for cerebral blood flow may be defined by:

$${AGI}_{CBF} = 100 * \frac{{CBFI}_{max} - {CBFI}_{0}}{{CBFI}_{0} * {T}_{AG}} \qquad ( Eqn . 6 )$$

Similarly, the AGI for cerebral blood volume may be defined by:

$${AGI}_{CBV} = 100 * \frac{{CBVI}_{max} - {CBVI}_{0}}{{CBVI}_{0} * {T}_{AG}} \qquad ( Eqn . 7 )$$

In some examples, the flow to volume ratio may be an extracted feature used to predict stroke risk. The flow to volume ratio may be determined based on the ratio of the AGI for cerebral blood flow to the AGI for cerebral blood volume:

$$\frac{{AGI}_{CBF}}{{AGI}_{CBV}} \qquad ( Eqn . 8 )$$

In one example, extracted features used to predict stroke risk include a growth factor of cerebral blood flow (τ_{growth}) and/or decay factor of cerebral blood flow (τ_{decay}). τ_{growth} may be extracted from cerebral blood flow and/or cerebral blood flow volume metrics data based on following equation.

$${f}_{1} = 1 + {CBFI}_{max} {e}^{\frac{{T}_{i} - {T}_{start}}{{τ}_{growth}}} \qquad ( Eqn . 9 )$$

The growth factor of cerebral blood flow (τ_{growth}) refers to the speed growth at which blood flow changes during hypercapnia or breath retention exercise. τ_{decay} may be extracted from cerebral blood flow and/or cerebral blood flow volume metrics data based on following equation.

$${f}_{2} = 1 + {CBFI}_{max} {e}^{\frac{{T}_{j} - {T}_{max}}{{τ}_{decay}}} \qquad ( Eqn . 10 )$$

The decay factor of cerebral blood flow (τ_{decay}) refers to the speed growth at which blood flow return to baseline after hypercapnia or breath retention exercise. Tₘₐₓ refers to a time after the brain stress time period at which the cerebral blood flow is maximal. Tᵢ are the times from the start time of the brain stress time period until the Tₘₐₓ. Tⱼ are the times from Tₘₐₓ until the cerebral brain flow returns to the baseline level.

In various examples, the physiological metric(s) data is taken during a period of a brain stress test such as breath-holding, inhaling air mixture with predefined CO₂ concentration, or another hypercapnic challenge. For example, the physiological metrics data may include breath-holding data or administering CO₂ gas mixture data.

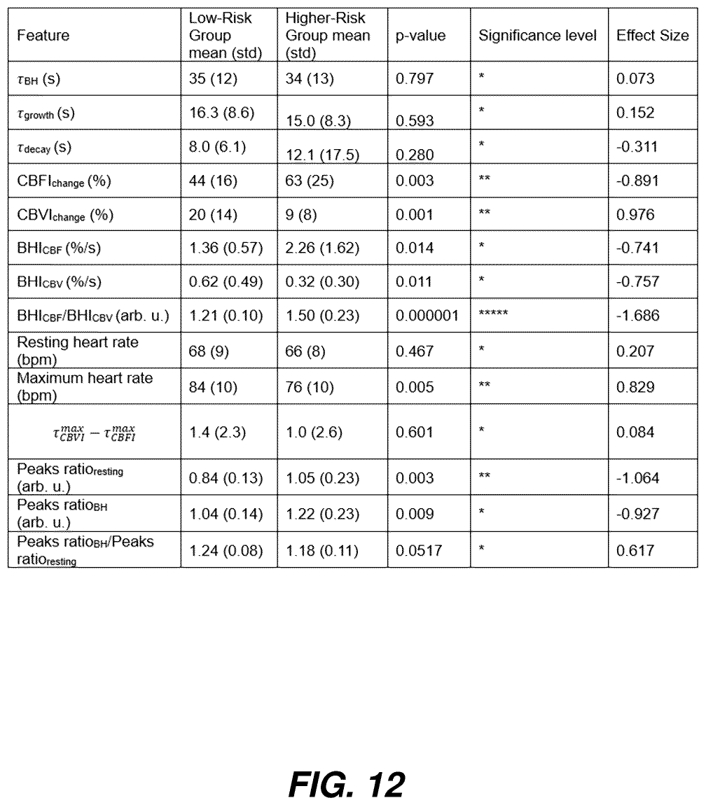

FIG. 12 depicts a table listing examples of features extracted from experimental physiological metric(s) data obtained by the apparatus 400 shown in FIG. 4 during a breath-holding test, according to various implementations. The experimental data depicted in FIG. 12 was obtained for 50 patients. The patients were divided into a “low risk” group and a “higher risk” group based on stroke risk scores, determined based on stroke questionnaire data. The mean and standard deviation of the stroke risk scores for the low-risk group were 1 and 0 respectively. The mean and standard deviation of the stroke risk scores for the high-risk group were 5 and 1 respectively. An SCOS system was used to obtain the data. The illustrated table includes the mean and standard deviation experimental data of the features for the low-risk group and the high-risk group, the p-values, significance level and effect size. The significance levels for single variable metrics were determined as follow: * (lowest significance) for p≥0.0063, ** for p<0.0063, *** for p<0.00133, **** for p<0.00013, and ***** (highest significance) for p<0.000013. The significance levels for two variable metrics were determined as follow: * (lowest significance) for p≥0.0018, ** for p<0.0018, *** for p<0.0004, **** for p<0.00004, and ***** (highest significance) for p<0.000004.

### Stroke Assessment Using CO₂ Gas Mixture Data

In some implementations, cerebral blood flow metrics information may be obtained during a time period that includes time that a patient or other user is administered a gas (air) mixture with a predefined concentration of CO₂ for inhaling in order to stimulate hypercapnia (i.e. excess carbon dioxide in the blood). For example, the gas mixture may be delivered through a pipeline system or gas cylinders to a facemask or nasal cannula placed in contact with the user's air passages to allow the user to inhale the gas mixture. The output gas mixture may be administered to the user over an administering time period (e.g., 30 seconds, 1 minute, 1-5 minutes, etc.) during which the user can inhale the gas mixture. In some cases, the CO₂ concentration of the gas mixture may be kept at a substantially constant value during the administering time period (e.g., CO₂ concentrations of about 1%, about 2%, about 3%, about 4%, or about 5%). In one case, the gas mixture administered to the user may be 5% CO₂, 20% O₂, and 75% N₂. In other cases, the concentration of CO₂ may vary over the administering time period and may be maintained below a maximum value (e.g., below 5%, below 6%, or below 4%). In various aspects, the CO₂ concentration of the gas mixture administered to the patient or other user is less than 5% during the administered time period.

Techniques for assessing stroke risk that involve obtaining cerebral blood flow metrics information during a time period that includes time that the user is administered an air mixture with a predefined CO₂ concentration may have advantages over other techniques that involve stimulating hypercapnia. For example, these techniques that administered an air mixture with a predefined CO₂ concentration may be more controllable and repeatable. With these techniques, equipment may be used to regulate the concentration of CO₂ in the air mixture, the duration of administering the gas mixture, and the flow of gas mixture delivered to the patient while the patient can breathe normally. These techniques do not require actions from the user and can be consistently applied. Moreover, these techniques can be used on unconscious patients.

In these implementations, cerebral blood metrics information (e.g., CBV information, CBF information, CBO information, etc.) may be obtained over a period of time, where, during a subset of the period of time, the patient is administered a gas mixture with a predefined concentration of carbon dioxide. Cerebral blood metrics information may also be obtained over a baseline time period, generally referring to the time period during which cerebral blood metrics are obtained prior to administering the gas mixture. Cerebral blood metrics information may also be obtained during a recovery time period, which generally begins at a time from when administering the gas mixture ends. In some cases, the cerebral blood metrics information may be obtained over a time period that spans the baseline time period, the administering time period, and the recovery time period.

Each of these time periods may be any suitable duration of time. Some examples of suitable durations of time for administering the gas mixture include 30 seconds, 45 seconds, 60 seconds, 2 minutes, 3 minutes, 4 minutes, 5 minutes, etc. In some implementations, the baseline time period and/or the recovery time period may be at least as long as the administering the gas mixture time period. In some embodiments, the recovery time period may be a time duration long enough that cerebral blood flow metrics return to within a predetermined range of the corresponding values during the baseline time period. In some such embodiments, the recovery time period may be dynamically adjusted. For example, the recovery time period may be stopped responsive to determined that cerebral blood metrics have returned to baseline values.

In certain cases, cerebral blood flow and cerebral blood volume may be determined using light emitted at a single wavelength (e.g., in the infrared wavelength region) and the captured reflected light data at the single wavelength. Cerebral blood flow may be determined based on speckle decorrelation time. Cerebral blood volume may be determined based on intensity of the reflected light signal. In certain cases, cerebral blood oxygenation may be determined based on the ratio of reflected light at two different wavelengths (e.g., in instances in which light is emitted by at least two light sources at two different wavelengths, such as an infrared wavelength and a near infrared wavelength).

FIG. 13 is a block diagram of an example system 1300 for determining stroke likelihood data based on data obtained during a time period in which air with a predefined CO₂ concentration or range of CO₂ concentrations is administered to a patient (referred to herein as “administering CO₂ gas mixture data”), in accordance with some embodiments. Some of the elements shown in FIG. 13 are similar or analogous to elements shown in FIG. 1. For the sake of brevity, the prior discussion of such similar or analogous elements with regard to FIG. 1 may be assumed to be equally applicable, unless indicated otherwise in the following discussion, to the similar or analogous counterparts of those elements in FIG. 13 that share the same last two digits in their respective callouts as in FIG. 1.

As illustrated in FIG. 13, system 1300 includes a stroke prediction engine 1302. In some implementations, stroke prediction engine 1302 may include a trained machine learning model or other computational model configured to take, as input, administering CO₂ gas mixture data 1304, and generate, as an output, stroke likelihood data 1306. The trained machine learning model may be a perceptron, a random forest, a deep neural network (DNN), or any other suitable architecture. In instances in which CO₂ gas mixture data 1304 includes raw cerebral blood metric data (e.g., time traces of cerebral blood flow, cerebral blood volume, and/or cerebral blood oxygenation, as shown in and described below in connection with FIG. 7), the trained machine learning model may be a DNN. Such a DNN may be able to identify features in the cerebral blood metric data not observable or identifiable by a human that are useful for predicting stroke risk.

In other embodiments, in instances in which administering CO₂ gas mixture data 1304 includes extracted features of cerebral blood metric data, (as described in more detail in connection with FIGS. 7 and 8) the trained machine learning model may be a perceptron, a random forest, or other type of architecture configured to take extracted features as input and generate a stroke likelihood. Note that the administering CO₂ gas mixture data may be obtained using light emitted into the brain of the patient using one or more light sources and using data representative of absorbed/reflected light using one or more light detectors. Such light emitters and light detectors may be disposed on a headband, as shown in and described in more detail in connection with FIGS. 2A and 2B and FIG. 4.

Stroke likelihood data 1306 may be a number on a discrete scale, a continuous scale, or the like. Stroke likelihood data 1306 may represent a likelihood that, given the cerebral blood metrics represented in administering CO₂ gas mixture data 1304, the patient will have a stroke within a predetermined future time period (e.g., within the next year, within the next five years, within the lifetime of the patient, etc.).

It should be noted that stroke prediction engine 1302 may be implemented by one or more computing devices or one or more processors. For example, such a computing device and/or processor may be configured to analyze data from one or more light detectors, generate cerebral blood flow metrics, and/or provide data representative of the cerebral blood flow metrics to a trained machine learning model to generate stroke likelihood data 1306. In some implementations, the one or more computing device and/or one or more processors may be disposed on the same headset as the one or more light emitters and one or more light detectors. Additionally, or alternatively, the one or more computing devices and/or one or more processors may be communicatively coupled to the headset, e.g., by a wireless or wired communication channel.

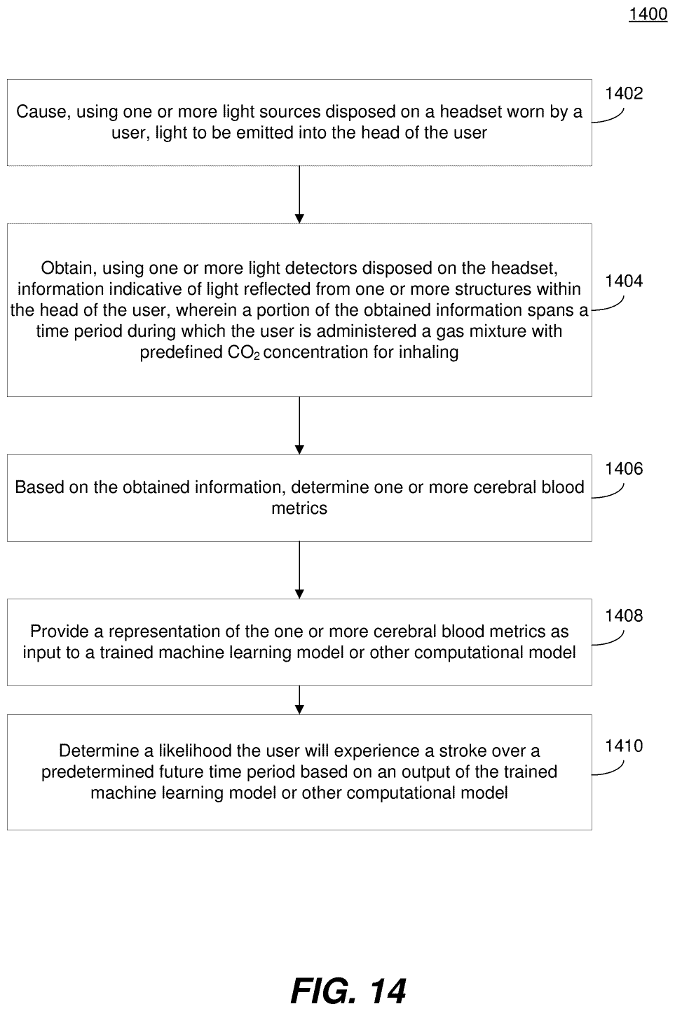

FIG. 14 is a flowchart of an example process 1400 for determining a stroke likelihood using cerebral blood metrics in accordance with some embodiments. Some of the elements shown in FIG. 14 are similar or analogous to elements shown in FIG. 9. For the sake of brevity, the prior discussion of such similar or analogous elements with regard to FIG. 9 may be assumed to be equally applicable, unless indicated otherwise in the following discussion, to the similar or analogous counterparts of those elements in FIG. 14 that share the same last two digits in their respective callouts as in FIG. 9.

Blocks of process 1400 may be executed by one or more processors of one or more computing devices (e.g., computing device in FIG. 21). In some embodiments, at least one of the one or more computing devices may be disposed on a headband on which the one or more light sources and light detectors are also disposed. Accordingly, in some such embodiments, cerebral blood metrics, extracted features associated with the cerebral blood metrics, and/or the stroke likelihood may be determined by the computing device itself disposed on the apparatus. Alternatively, in some embodiments, data obtained by the one or more light detectors, and/or data representative of the cerebral blood metrics may be transmitted from a computing device disposed on the headband or other components of the headset to a second computing device remote from or separate from the headband, where the second computing device generates the stroke likelihood data. In some implementations, blocks of process 1400 may be executed in an order other than what is shown in FIG. 14. In some embodiments, one or more blocks of process 1400 may be omitted, and/or two or more blocks may be executed substantially in parallel.

Process 1400 can begin at 1402 by causing, using one or more light sources disposed on a headband on a headset worn by a user, light to be emitted into the head of the user. Examples of headsets or headbands are shown in and described above in connection with FIGS. 2A and 2B and FIGS. 4 and 5. The one or more light sources may include one or more lasers, one or more LEDs, etc. As described above, in some implementations, two light sources of different types (e.g., a laser and an LED), each of which may emit light in a different wavelength region (e.g., infrared and near infrared) may be packaged together as a light emission package. In some implementations, multiple light emission packages may be disposed on a headband, each configured to emit light into different regions of the user's head or brain. Note that light may be emitted continuously, or may be pulsed.

At 1404, process 1400 may obtain, using one or more light detectors disposed on a headband, information indicative of light reflected from one or more structures within the head or brain of the user, wherein a portion of the obtained information spans an administering time period during which a gas mixture with a predefined concentration of CO₂ is administered to the user for inhaling via a face mask, tubing, or the like. In some cases, the obtained light reflection data may span a baseline time period, the administering time period, and a recovery time period.

Examples of one or more light detectors (e.g., cameras, photodetectors, etc.) disposed on headbands are shown in and described above in connection with FIGS. 2A and 2B and FIGS. 4 and 5. In instances in which a light emission package includes two light sources each emitting light in a different wavelength range, a corresponding light detection package may include two light detectors, each configured to receive reflected light corresponding to emissions from the corresponding light emitter. In one example, light emitted by a laser may be reflected from various head and brain structures and may be captured by a camera, and light emitted by an LED may be reflected and captured by a photodetector. In some cases, an apparatus may include multiple (e.g., two, four, eight, ten, etc.) light emission packages and corresponding light detection packages, the obtained information may correspond to different brain regions (e.g., a left frontal lobe region, a right frontal lobe region, a left parietal lobe region, a right parietal lobe region, etc.).

At 1406, process 1400 can, based on the obtained information, determine one or more cerebral blood metrics. As described above, the cerebral blood metrics may include cerebral blood volume, cerebral blood flow, and/or cerebral blood oxygenation. Process 1400 may additionally determine a duration of time the user is administered the gas mixture with the predefined CO₂ concentration. The duration of time may be determined by, e.g., thresholding any of the cerebral blood metrics to determine a time point at which the cerebral blood metric began deviating from baseline to the time point at which the cerebral blood metric reached a minimum or maximum value. Note that, as described above, cerebral blood volume may be determined based on intensity of the reflected light signal at a single wavelength. Cerebral blood flow may be determined using DCS and/or SCOS (as shown in and described above in connection with FIG. 6). Cerebral blood oxygenation may be determined based on the ratio of reflected light at two different wavelengths (e.g., in instances in which light is emitted by at least two light sources at two different wavelengths, such as an infrared wavelength and a near infrared wavelength).

At 1408, process 1400 can provide a representation of the one or more cerebral blood metrics as input to a trained machine learning model or other computational model. Note that the representation of the one or more cerebral blood metrics may include the raw data of the cerebral blood metrics as a function of time, or, alternatively, may include one or more extracted features extracted from the one or more cerebral blood metrics. Examples of extracted features are described above.

At 1410, process 1400 can determine a likelihood the user will experience a stroke over a predetermined future time period based on an output of the trained machine learning model or other computational model. For example, the likelihood may correspond to a time period of the next year, the next five years, the next ten years, the remainder of their lifetime, etc. The likelihood of stroke may be provided as a discrete classification, a continuous value, or other suitable format. In some cases, the likelihood of stroke may be provided as a discrete classification of low risk, medium risk, high risk, etc. In other cases, the likelihood of stroke may be provided as an integer on a scale (e.g., an integer on a scale from 1-10, an integer on a scale from 1-7, etc.). The stroke likelihood may be presented in any suitable manner. Some examples of suitable manners are described herein.

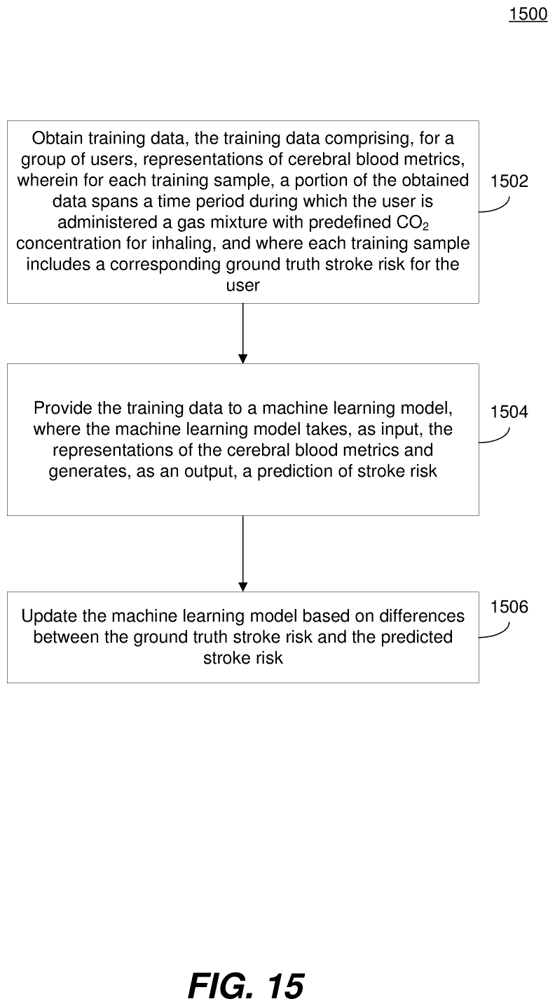

FIG. 15 is a flowchart of an example process 1500 for training a machine learning model in accordance with some embodiments. Some of the elements shown in FIG. 15 are similar or analogous to elements shown in FIG. 10. For the sake of brevity, the prior discussion of such similar or analogous elements with regard to FIG. 10 may be assumed to be equally applicable, unless indicated otherwise in the following discussion, to the similar or analogous counterparts of those elements in FIG. 15 that share the same last two digits in their respective callouts as in FIG. 15. Blocks of process 1500 may be executed by one or more computing devices. In some implementations, blocks of process 1500 may be executed in an order other than what is shown in FIG. 15. In some embodiments, two or more blocks of process 1500 may be executed substantially in parallel. In some embodiments, one or more blocks of process 1500 may be omitted.

Process 1500 can begin at 1502 by obtaining training data, the training data comprising, for a group of users, representations of cerebral blood metrics, wherein for each training sample, a portion of the obtained data spans a time period during which the user is administered a gas mixture with a predefined CO₂ concentration, and where each training sample includes a corresponding ground truth stroke risk for the user.

As described above, the representations of cerebral blood metrics may include one or more cerebral blood metrics (e.g., CBF, CBV, and/or CBO) as a function of time, and/or extracted features associated with the cerebral blood metrics and/or with the administering of the gas mixture with the predefined CO₂ concentration. Note that cerebral blood metrics for the training data may have been collected using one or more light sources and/or one or more light detectors disposed on a headset or headband similar to the one shown in and described above in connection with FIGS. 2A and 2B or FIGS. 4 and 5. The cerebral blood metrics may be determined based on collected light reflectance data as described above. Ground truth stroke risk data may be questionnaire based, or may be actual stroke occurrence data based on a longitudinal following of the users over time.

At 1504, process 1500 can provide the training data to a machine learning model, where the machine learning model takes, as input, the representations of the cerebral blood metrics and generates, as an output, a prediction of stroke risk.

At 1506, process 1500 can update the machine learning model based on differences between the ground truth stroke risk and the predicted stroke risk. For example, weights of the model may be updated based on a loss function that considers the difference between the ground truth stroke risk and the predicted stroke risk. Note that model updating may be performed for each training sample, or for a batch of training samples.

### Examples of Stroke Risk Scoring Techniques

According to certain aspects, techniques disclosed herein predict stroke risk scores. In some cases, the stroke risk score may be an integer on a scale from lowest risk to highest risk or visa versa. For example, a stroke risk score may be an integer between 1 (lowest risk) and 5 (highest risk), integer between 1 (lowest risk) and 10 (highest risk), integer between 1 (lowest risk) and 7 (highest risk), etc. As another example, the stroke risk score may be a number on a continuous scale (e.g., between 0 and 1, between 1 and 10, etc.) from lowest to highest risk or highest to lowest risk. The stroke risk score or a change in the risk score over time may represent a likelihood that, given the physiological metrics represented in the brain stress test data, the user will have a stroke within a predetermined future time period (e.g., within the next year, within the next five years, within the lifetime of the patient, etc.). Any suitable apparatus or multiple apparatus (e.g., apparatus 400 in FIG. 4, apparatus 200 in FIG. 2A, etc.) may be implemented to generate the cerebral blood metrics data used as input into the computational model to predict the stroke risk score. The stroke risk score may be presented in any suitable manner. For example, the apparatus 400 in FIG. 4 or the apparatus 200 in FIG. 2A may include a display for displaying the predicted stroke risk score to the healthcare provider.

Techniques that provide such physiology-based stroke risk scores may be advantageous in preventative and managed care of high-risk patients. In some cases, the stroke risk score may be automatically (e.g., without user input) stored in an electronic medical record or other record associated with a patient or other user. In cases in which the stroke likelihood is stored, e.g., as medical data, the stroke risk score may be stored in conjunction with timestamp information indicating a date and/or time the stroke likelihood prediction was made. This may allow a physician or other healthcare provider to monitor changes in stroke risk score for a given patient over time. This may allow the healthcare provider to determine whether various interventions are modifying the likelihood of stroke (e.g., reducing the risk of stroke) over time. In some implementations, an updated stroke risk score may be determined after a first stroke risk score is determined. A difference between the first risk score and the updated stroke risk score may be determined, e.g., to determine if the patient's likelihood of experiencing stroke remains the same over time, is increasing over time, or is decreasing over time. In some embodiments, a recommendation may be generated based on the change in stroke risk score over time. For example, a recommendation to continue or implement particular lifestyle modifications may be made, a recommendation to initiate a particular medical treatment may be made, etc.

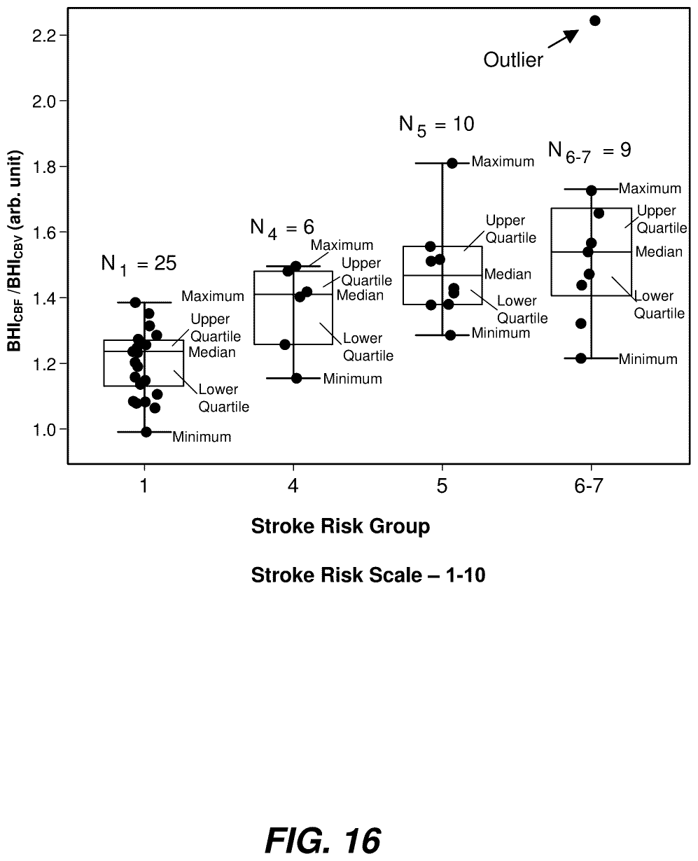

FIG. 16 illustrates experimental data in accordance with some embodiments. The experimental data depicted in FIG. 16 is based on data obtained from 41 patients. The cerebral blood flow (CBF) and cerebral blood volume (CBV) were obtained for forty one (41) patients during a breath-holding test. The CBF and CBV were determined using the light emittance and light reflectance techniques described above, in particular, using a laser emitting infrared light, and a camera configured to obtain reflected light. Cerebral blood flow was determined using SCOS, and cerebral blood volume was determined based on intensity of captured reflected light. The ratio of the percentage change of cerebral blood flow to the percentage change of cerebral blood volume (BHI_{CBF}/BHI_{CBV}) was determined as the BHI of cerebral blood flow to the BHI of cerebral blood volume. For the data shown, the percentage change was measured from the peak after initiation of breath holding to a baseline value using the BHI, as described above, for both cerebral blood flow and cerebral blood volume.

Using stroke questionnaire data, stroke risk scores for the patients were provided on a scale of 1-10. Based on the stroke risk scores, the patients were divided into four risk score groups: a first risk score group (risk score 1), a second risk score group (risk score 4), a third risk score group (risk score 5), and a third risk score group (risk score of 6 or 7).

FIG. 16 illustrates box plots of the ratio of the percentage change of cerebral blood flow to the percentage change of cerebral blood volume (BHI_{CBF}/BHI_{CBV}) for the four risk score groups in box plot form. As shown, the increasing trend in the percentage change of cerebral blood flow to the percentage change of cerebral blood volume (BHI_{CBF}/BHI_{CBV}) with increasing stroke risk scores shows correlation with the stroke questionnaire data. FIG. 16 also includes one instance of an abnormally high flow to volume ratio, BHI_{CBF}/BHI_{CBV} (labeled Outlier) in the fourth risk score group which may indicate a higher risk of stroke as compared to the other patients in the fourth risk score group. The data shows a statistically significant in BHI_{CBF}/BHI_{CBV} values between patients having different stroke risk scores, validating the use of BHI_{CBF}/BHI_{CBV} as a predicter of a stroke risk score.

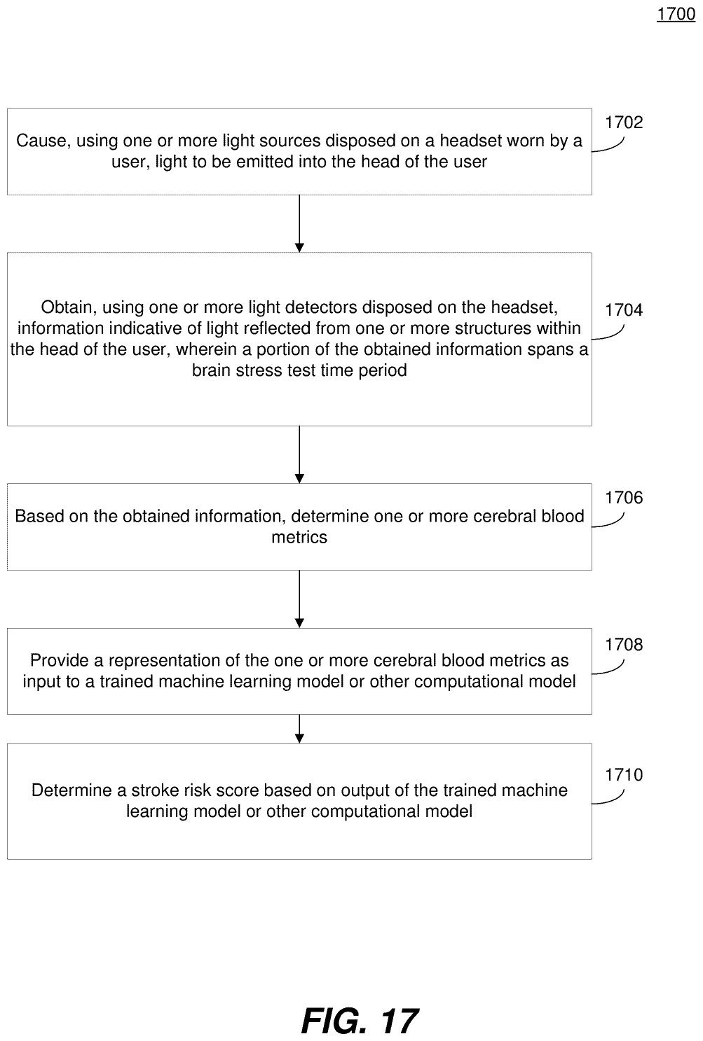

FIG. 17 is a flowchart of an example process 1700 for determining a stroke risk score using cerebral blood metrics in accordance with some embodiments. Blocks of process 1700 may be executed by one or more processors of one or more computing devices (e.g., computing device in FIG. 21). In some embodiments, at least one of the one or more computing devices may be disposed on an apparatus used to obtain cerebral blood metrics information. Accordingly, in some such embodiments, cerebral blood metrics, extracted features associated with the cerebral blood metrics, and/or the stroke likelihood may be determined by the computing device itself disposed on the apparatus. Alternatively, in some embodiments, data obtained may be transmitted from a computing device disposed on the apparatus to another computing device remote from or separate from the apparatus, where the second computing device generates the stroke risk score. In some implementations, blocks of process 1700 may be executed in an order other than what is shown in FIG. 17. In some embodiments, one or more blocks of process 1700 may be omitted, and/or two or more blocks may be executed substantially in parallel.

At 1702, process 1700 causes one or more light sources (e.g., include one or more lasers, one or more LEDs, etc.) disposed on a headband worn by a user to emit light into the head of the user. Examples of headsets or headbands are shown in and described above in connection with FIGS. 2A and 2B and FIGS. 4 and 5. In some cases, two light sources of different types (e.g., a laser and an LED), each of which may emit light in a different wavelength region (e.g., infrared and near infrared) may be packaged together as a light emission package. In some implementations, multiple light emission packages may be disposed on a headband, each configured to emit light into different regions of the user's head or brain. The light may be emitted continuously, or may be pulsed.

At 1704, process 1700 may obtain, using one or more light detectors disposed on the headband, information indicative of light reflected from one or more structures within the head of the user. At least a portion of the information may be obtained during a time period that spans a baseline time period, a brain stress test time period (e.g., breath-holding time period or administering gas mixture with predefined CO₂ concentration time period), and a recovery time period. In some cases, an apparatus may include multiple (e.g., two, four, eight, ten, etc.) light emission packages and corresponding light detection packages for obtaining information corresponding to different brain regions (e.g., a left frontal lobe region, a right frontal lobe region, a left parietal lobe region, a right parietal lobe region, etc.). In some cases, the information may include transcranial optical signals with speckle patterns and/or multispectral absorption.

At 1706, process 1700 can, based on the obtained information, determine one or more cerebral blood metrics. As described above, the cerebral blood metrics may include cerebral blood volume, cerebral blood flow, and/or cerebral blood oxygenation. Process 1700 may additionally determine a duration of the brain stress test. For example, the duration of time may be determined by, e.g., thresholding any of the cerebral blood metrics to determine a time point at which the cerebral blood metric began deviating from baseline to the time point at which the cerebral blood metric reached a minimum or maximum value. Note that, as described above, cerebral blood volume may be determined based on intensity of the reflected light signal at a single wavelength. Cerebral blood flow may be determined using DCS and/or SCOS (as shown in and described above in connection with FIG. 6). Cerebral blood oxygenation may be determined based on the ratio of reflected light at two different wavelengths (e.g., in instances in which light is emitted by at least two light sources at two different wavelengths, such as an infrared wavelength and a near infrared wavelength).

At 1708, process 1700 can provide a representation of the one or more cerebral blood metrics as input to a trained machine learning model or other computational model. Note that the representation of the one or more cerebral blood metrics may include the raw data of the cerebral blood metrics as a function of time (e.g., time traces of CBV or CBF), or, alternatively, may include one or more features extracted from the one or more cerebral blood metrics. Examples of extracted features are described above.

At 1710, process 1700 can determine a stroke risk score based on output of the trained machine learning model or other computational model. For example, a trained machine learning model or other computational model may use, as input, time traces or one or more extracted features (e.g., BHI_{CBF}/BHI_{CBV} determined as the BHI of cerebral blood flow to the BHI of cerebral blood volume) and output, a stroke risk score. The stroke risk score may be indicative of a likelihood the user will experience a stroke over a predetermined future time period such as a time period of the next year, the next five years, the next ten years, the remainder of their lifetime, etc. The stroke risk score may be provided as integer on a scale (e.g., scale of 1-10, scale of 1-7, etc.).

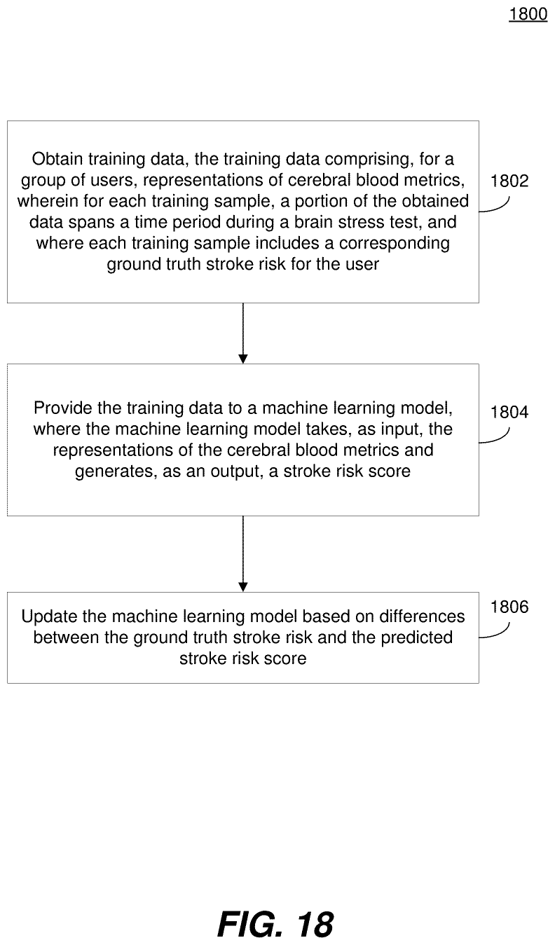

FIG. 18 is a flowchart of an example process 1800 for training a machine learning model in accordance with some embodiments. Some of the elements shown in FIG. 18 are similar or analogous to elements shown in FIG. 10. For the sake of brevity, the prior discussion of such similar or analogous elements with regard to FIG. 10 may be assumed to be equally applicable, unless indicated otherwise in the following discussion, to the similar or analogous counterparts of those elements in FIG. 18 that share the same last two digits in their respective callouts as in FIG. 18. Blocks of process 1800 may be executed by one or more computing devices. In some implementations, blocks of process 1800 may be executed in an order other than what is shown in FIG. 18. In some embodiments, two or more blocks of process 1800 may be executed substantially in parallel. In some embodiments, one or more blocks of process 1800 may be omitted.

Process 1800 can begin at 1802 by obtaining training data, the training data comprising, for a group of users, representations of cerebral blood metrics, wherein for each training sample, a portion of the obtained data spans a time period during which the user is administered the brain stress test, and where each training sample includes a corresponding ground truth stroke risk for the user. The representations of cerebral blood metrics may include raw cerebral blood metrics data (e.g., time traces of cerebral blood flow, cerebral blood volume, and/or cerebral blood oxygenation), and/or extracted features associated with the cerebral blood metrics. In some embodiments, ground truth data may be questionnaire data, where the questionnaire predicts a stroke likelihood based on demographic data, lifestyle data, physiological metrics (e.g., blood pressure, resting heart rate, etc.), and the like. Alternatively, in some embodiments, ground truth data may be actual stroke occurrence data, e.g., obtained from longitudinal data that collects representations of cerebral blood metrics from a set of patients who are followed over time.

At 1804, process 1800 can provide the training data to the machine learning model, where the machine learning model takes, as input, the representations of the cerebral blood metrics and generates, as an output, a stroke risk score. The machine learning model may be trained by providing the representations of cerebral blood flow metrics as input, obtaining a stroke risk score based on the input, and updating weights of the machine learning model based on a difference between the predicted stroke risk and the ground truth stroke risk score. This procedure may be implemented to train a DNN that operates on time traces of cerebral blood metrics as a function of time, and/or a perceptron, random forest, or other type of network that operates on extracted features associated with cerebral blood metrics.

At 1806, process 1800 can update the weights of the machine learning model based on differences between the ground truth stroke risk and the predicted stroke risk. For example, weights of the model may be updated based on a loss function that considers the difference between the ground truth stroke risk and the predicted stroke risk score. Note that model updating may be performed for each training sample, or for a batch of training samples.

In one implementation, the process may also include testing/validating the trained machine learning model using validation data including, for a test group of users, representations of cerebral blood metrics. The machine learning model may be validated by providing the representations of cerebral blood flow metrics for the test group of users as input, obtaining a stroke risk scores based on the input, and comparing the predicted stroke risk scores with corresponding ground truth stroke risk scores.

In one implementation, the process may also include tracking the stroke risk scores output from the trained machine learning model over time (e.g., over one month, over two months, over two years, etc.) to determine the progression of the stroke risk scores.

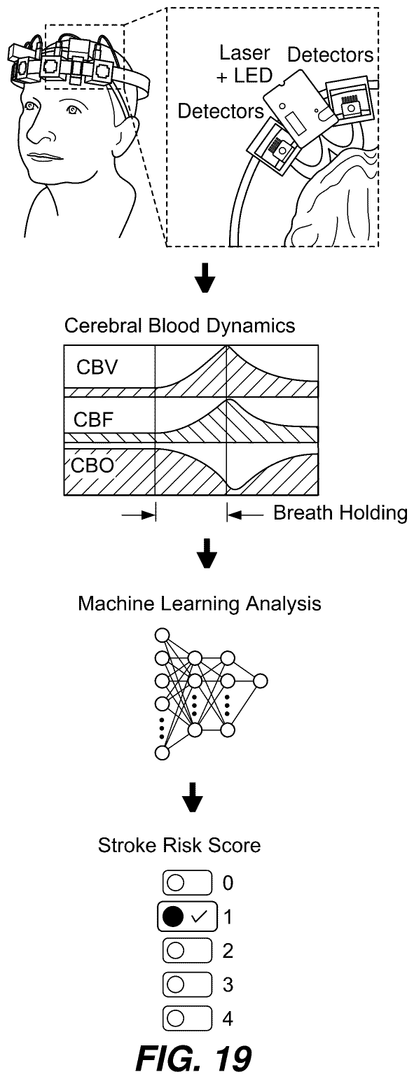

FIG. 19 is a diagram depicting an example of a process for determining a stroke risk score, according to some embodiments. As shown, the process provides, as input, time traces of cerebral blood metrics including CBF, CBV, and CBO, to a trained machine learning model. The trained machine learning model generates, as output, stroke likelihood data that can be used to generate a stroke risk score. The machine learning model may be trained using the process shown in and described with reference to FIG. 18. In one implementation, the trained learning model is a DNN. The machine learning model may be a DNN or other suitable architecture for accepting raw data traces.

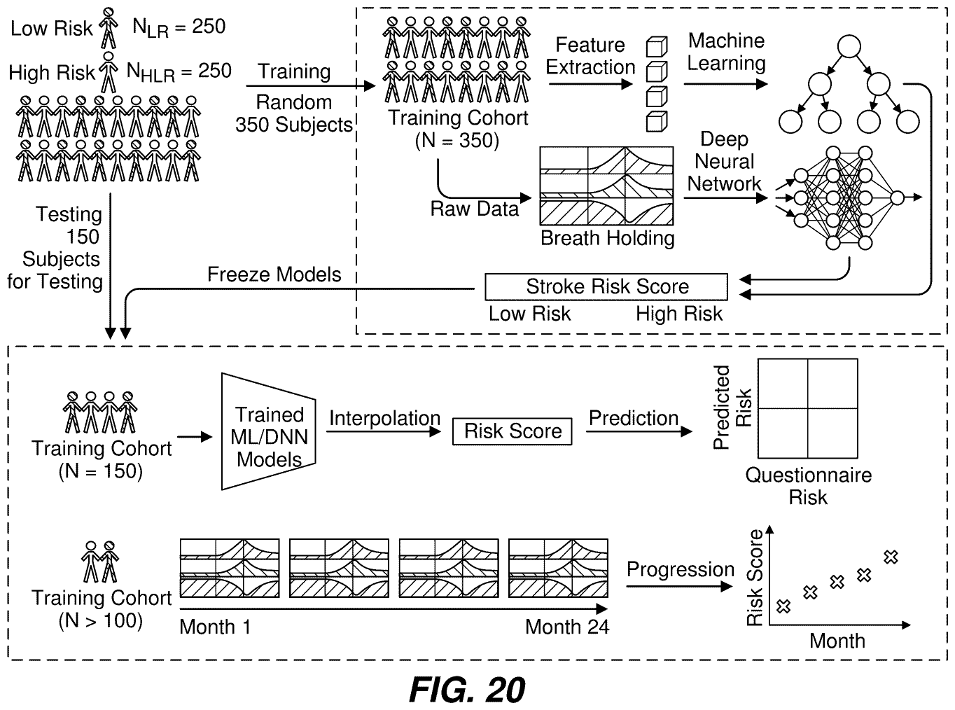

FIG. 20 is a diagram depicting an example of a process for training and testing one or more machine learning models that generate, as output, stroke likelihood data that includes or can be interpolated to generate stroke risk scores, according to some embodiments. The process obtains training data, for a group of users, that includes representations of cerebral blood metrics, wherein for each training sample, a portion of the obtained data spans a time period during a brain stress test, and where each training sample includes a corresponding ground truth stroke risk for the user. In one implementation, one or more features are extracted from the training data and the features are used as input to train a classifier machine learning model such as a perceptron, a random forest, or other suitable classifier architecture. In one implementation, the raw training data (e.g., time traces of CBF, CBV, and/or CBO) are used as input to train a deep neural network (DNN) or other suitable architecture for accepting raw data. The machine learning model(s) may be trained by providing the training data as input, obtaining a stroke risk score based on the input, and updating weights of the machine learning model(s) based on a difference between the predicted stroke risk score and the ground truth stroke risk score. In some embodiments, the ground truth stroke risk score may be derived from questionnaire data, where the questionnaire predicts a stroke risk score based on demographic data, lifestyle data, physiological metrics, and the like. Alternatively, in some embodiments, the ground truth risk score may be based on actual stroke occurrence data, e.g., obtained from longitudinal data that collects representations of cerebral blood metrics from a set of patients who are followed over time. The illustrated process also includes testing/validating the machine learning model(s) using validation data including, for a test group of users, representations of cerebral blood metrics. The machine learning model may be validated by providing the representations of cerebral blood flow metrics for the test group of users, as input, obtaining stroke risk scores based on the input, and comparing the predicted stroke risk scores with corresponding ground truth stroke risk scores. The process may also track stroke risk scores output from the trained machine learning model(s) over time (e.g., over one month, over two months, over two years, etc.) to determine the progression of the stroke risk scores.

### Other Systems for Obtaining Cerebral Blood Metrics Information

In various embodiments, the cerebral blood metrics information is obtained by an apparatus with one or more light sources (e.g., one or more lasers, one or more LEDs, etc.) and one or more light detectors disposed on a headband worn on the head of the user. Examples of headsets or headbands are shown in and described above in connection with FIGS. 2A and 2B and FIGS. 4 and 5. In some cases, the apparatus includes two light sources of different types (e.g., a laser and an LED), each of which may emit light in a different wavelength region (e.g., infrared and near infrared) may be packaged together as a light emission package. In some implementations, multiple light emission packages may be disposed on a headband, each configured to emit light into different regions of the user's head or brain. In this aspect, the process may cause the one or more light sources to emit light into the head of the user and using the one or more light detectors obtain information indicative of light reflected from one or more structures within the head. A portion of the obtained information may be obtained during a time period that spans a baseline time period, a brain stress test time period (e.g., breath-holding time period or administering gas mixture with predefined CO₂ concentration time period), and a recovery time period. In other embodiments, one or more other types of apparatus may be used to obtain the cerebral blood metrics information such as near-infrared spectroscopy (NIRS), diffuse correlation spectroscopy (DCS), diffuse wave spectroscopy (DWS), diffuse speckle contrast flowmetry (DSCF), interferometric speckle visibility spectroscopy (iSVS), or transcranial doppler ultrasound (TCD).

### Computational Systems

The techniques described above may be implemented using one or more computing devices. For example, a machine learning model may be trained and/or utilized at inference time using a computational device such as a server device, a laptop computer, a desktop computer, or the like. FIG. 21 illustrates an example computing device that may be used, e.g., to implement blocks of process 900 and/or 1000 of FIGS. 9 and/or 10, respectively. Note that such a computing device may be part of a headset or headband comprising one or more light sources and/or one or more light detectors (e.g., the computing device may be disposed on a portion of the headset or headband), or may be communicatively coupled to the headset or headband (e.g., via a wireless communication channel, such as BLUETOOTH).

In FIG. 21, the computing device(s) 2150 includes one or more processors 2160 (e.g., microprocessors), a non-transitory computer readable medium (CRM) 2170 in communication with the processor(s) 2160, and one or more displays 2180 also in communication with processor(s) 2160.

Processor(s) 2160 is in electronic communication with CRM 2170 (e.g., memory). Processor(s) 2160 is also in electronic communication with display(s) 2180, e.g., to display image data, text, etc. on display 2180.

Processor(s) 2160 may retrieve and execute instructions stored on the CRM 2170 to perform one or more functions described above. For example, processor(s) 2160 may execute instructions to perform one or more operations to analyze collected data (e.g., light reflection/absorption data), provide collected data to a trained model, train a machine learning model to generate a stroke risk prediction, etc.

The CRM (e.g., memory) 2170 can store instructions for performing one or more functions of the described above. These instructions may be executable by processor(s) 2170. CRM 2170 can also store raw images, e.g., speckle images, or the like.

### Example Embodiments:

Embodiment 1: A method of determining stroke risk, the method comprising: causing, using one or more light sources disposed on a headset worn by a user, light to be emitted into the head of the user; obtaining, using one or more light detectors disposed on the headset, information indicative of light reflected from one more structures within the head of the user, wherein a portion of the obtained information spans a time period during which the user was holding their breath; based on the obtained information, determining one or more cerebral blood metrics; providing a representation of the one or more cerebral blood metrics as input to a trained machine learning model; and determining a likelihood the user will experience a stroke over a predetermined future time period based on an output of the trained machine learning model.
Embodiment 2: The method of embodiment 1, further comprising: determining an updated likelihood the user will experience a stroke based on updated cerebral blood metrics; and determining a change between the likelihood and the updated likelihood.
Embodiment 3: The method of embodiment 2, further comprising providing at least one recommendation based on the change between the likelihood and the updated likelihood.
Embodiment 4: The method of any one of embodiments 1-3, wherein the one or more light sources comprise at least two light sources, each configured to emit light in a different wavelength.
Embodiment 5: The method of claim 4, wherein the at least two light sources comprise a laser and a light emitting diode packaged together.
Embodiment 6: The method of any one of embodiments 4 or 5, wherein the at least two light sources comprise a laser configured to emit light in an infrared wavelength range.
Embodiment 7: The method of any one of embodiments 4-6, wherein the at least two light sources comprise a light emitting diode configured to emit light in a near-infrared wavelength range.
Embodiment 8: The method of any one of embodiments 1-7, wherein the one or more light detectors comprise at least two detectors of different types.
Embodiment 9: The method of embodiment 8, wherein the at least two detectors of different types comprise at least one camera.
Embodiment 10: The method of embodiment 9, wherein the at least one camera is configured to capture images comprising a speckle pattern.
Embodiment 11: The method of any one of embodiments 1-10, wherein the one or more cerebral blood metrics comprise at least one of: cerebral blood flow, cerebral blood oxygenation, or cerebral blood volume.
Embodiment 12: The method of embodiment 11, wherein the cerebral blood flow is determined based on a decorrelation time associated with a series of speckle patterns obtained from a series of images captured by at least one camera included in the one or more light detectors.
Embodiment 13: The method of any one of embodiments 11 or 12, wherein the representation of the obtained information comprises a ratio of change in cerebral blood flow during the time period the user was holding their breath to a baseline period to a change in cerebral blood volume during the time period the user was holding their breath to a baseline period.
Embodiment 14: The method of any one of embodiments 1-13, wherein providing the representation of the one or more cerebral blood metrics as input comprises providing raw traces of the one or more cerebral blood metrics as a function of time to the trained machine learning model, and wherein the trained machine learning model is a deep neural network (DNN).
Embodiment 15: The method of any one of embodiments 1-14, wherein providing the representation of the one or more cerebral blood metrics as input comprises providing features of the one or more cerebral blood metrics as an input to trained machine learning model.
Embodiment 16: A system for determining stroke risk, the system comprising: a headband configured to encircle a head of a wearer of a headset; a plurality of light sources attached to the headband; a plurality of light detectors attached to the headband; and one or more processors. The one or more processors may be configured to: cause, using the one or more light sources, light to be emitted into the head of the wearer; obtain, using the one or more light detectors, information indicative of light reflected from one more structures within the head of the wearer, wherein a portion of the obtained information spans a time period during which the wearer was holding their breath; based on the obtained information, determine one or more cerebral blood metrics; determine a likelihood the wearer will experience a stroke over a predetermined future time period based on the one or more cerebral blood metrics.
Embodiment 17: The system of embodiment 16, wherein the plurality of light sources comprise a plurality of light emission packages, each light emission package comprising at least two light sources configured to emit light in different wavelengths.
Embodiment 18: The system of embodiment 17, wherein the at least two light sources comprise a laser configured to emit light in an infrared wavelength range, and a light emitting diode (LED) configured to emit light in a near infrared wavelength range.
Embodiment 19: The system of any one of embodiments 16-18, wherein the plurality of light detectors comprise a plurality of light detection packages, each light detection package comprising at least two light detectors.
Embodiment 20: The system of embodiment 19, wherein a light detector of the at least two light detectors of a light detection package comprises a camera.
Embodiment 21: The system of embodiment 20, wherein the obtained information comprises a speckle pattern obtained using images captured by the camera.
Embodiment 22: The system of embodiment 21, wherein the one or more cerebral blood metrics comprise a cerebral blood flow determined based on the speckle pattern.
Embodiment 23: The system of any one of embodiments 16-22, wherein a distance between a light source of the one or more light sources and a light detector of the one or more light detectors is adjustable by changing a position of the light source and/or a light detector on the headband.
Embodiment 24: A method of determining stroke risk, the method comprising: causing, using one or more light sources disposed on a headset worn by a user, light to be emitted into the head of the user; obtaining, using one or more light detectors disposed on the headset, information indicative of light reflected from one more structures within the head of the user, wherein a portion of the obtained information spans a time period during which the user was holding their breath; based on the obtained information, determining a cerebral blood flow as a function of time and a cerebral blood volume as a function of time, wherein the cerebral blood flow and the cerebral blood volume include the time period during which the user was holding their breath and a baseline time period before the user was holding their breath; and determining a likelihood the user will experience a stroke over a predetermined future time period based on the cerebral blood flow and the cerebral blood volume.
Embodiment 25: The method of embodiment 24, wherein the likelihood the user will experience the stroke is based on a ratio of a percentage change of cerebral blood flow from a peak after the user began holding their breath to a baseline cerebral blood flow during the baseline time period to a percentage change of cerebral blood volume from a peak after the user began holding their breath to a baseline cerebral blood volume during the baseline time period.
Embodiment 26: A method of training a machine learning model to predict stroke risk, the method comprising: obtaining training data, the training data comprising, for a group of users, representations of cerebral blood metrics, wherein for each training sample, a portion of the obtained data spans a time period during which the user was holding their breath, and wherein each training sample includes a corresponding ground truth stroke risk for the user; providing the training data to a machine learning model, wherein the machine learning model takes, as input, the representations of the cerebral blood metrics and generates, as an output, a prediction of stroke risk; and updating the machine learning model based on differences between the ground truth stroke risk and the predicted stroke risk to generate a trained machine learning model configured to predict stroke risk.
Embodiment 27: The method of embodiment 26, wherein the representations of cerebral blood metrics are determined based on light reflectance data obtained using one or more light emitters and one or more light detectors disposed on a first head-worn device worn by users in the group of users, and wherein the trained machine learning model is provided to a computing device of a second head-worn device on which one or more light emitters and one or more light detectors are disposed.
Embodiment 28: The method of any one of embodiments 26 or 27, wherein the ground truth stroke risk is obtained based on questionnaire data.
Embodiment 29: The method of any one of embodiments 26-28, wherein the ground truth stroke risk is obtained based on longitudinal stroke occurrence data for users of the group of users.
Embodiment 30: A method of determining stroke risk, the method comprising: causing, using one or more light sources disposed on a headset worn by a user, light to be emitted into a head of the user; obtaining, using one or more light detectors disposed on the headset, information indicative of light reflected from one more structures within the head of the user, wherein at least a portion of the obtained information is obtained during a time period of hypercapnic stimulation; based on the obtained information, determining a representation of one or more cerebral blood metrics; providing the representation of the one or more cerebral blood metrics as input to a computational model; and determining a stroke risk score based on output of the computational model.
Embodiment 31: The method of embodiment 30, wherein the stroke risk score corresponds to a likelihood the user will experience a stroke over a predetermined future time period.
Embodiment 32: The method of embodiment 30, wherein the computational model is a trained machine learning model.
Embodiment 33: The method of embodiment 32, wherein the trained machine learning model comprises a deep neural network or a random forest classifier.
Embodiment 34: The method of embodiment 30, wherein another portion of the obtained information is obtained during a baseline time period before or after the time period of hypercapnic stimulation.
Embodiment 35: The method of embodiment 30, wherein the stroke risk score is an integer on a scale.
Embodiment 36: The method of embodiment 30, wherein during at least a portion of the time period of hypercapnic stimulation the user was (i) holding their breath or (ii) administered air with a concentration of carbon dioxide (CO₂).
Embodiment 37: The method of embodiment 30, wherein during at least a portion of the time period of hypercapnic stimulation the user was (i) holding their breath or (ii) administered air with a concentration of carbon dioxide (CO₂).
Embodiment 38: The method of embodiment 37, wherein the concentration of CO₂ (a) less than 5%, (b) between 1% and 5%, (c) between 1% and 10%.
Embodiment 39: The method of embodiment 30, further comprising: determining an updated stroke risk score based on updated cerebral blood metrics; and determining a change between the stroke risk score and the updated stroke risk score.
Embodiment 40: The method of embodiment 39, further comprising: providing at least one recommendation based on the change between the stroke risk score and the updated stroke risk score.
Embodiment 41: The method of embodiment 30, wherein the one or more light sources comprise at least two light sources, each configured to emit light in a different wavelength.
Embodiment 42: The method of embodiment 30, wherein the one or more cerebral blood metrics comprise at least one of: cerebral blood flow, cerebral blood oxygenation, or cerebral blood volume, percentage change in cerebral blood flow.
Embodiment 43: The method of embodiment 42, wherein the cerebral blood flow is determined based on a decorrelation time associated with a series of speckle patterns obtained from a series of images captured by the one or more light detectors.
Embodiment 44: The method of embodiment 30, wherein the representation of one or more cerebral blood metrics comprises a ratio of percentage change in cerebral blood flow from a peak during the time period of hypercapnic stimulation to a baseline cerebral blood volume during a baseline time period.
Embodiment 45: The method of embodiment 30, wherein the representation of the one or more cerebral blood metrics comprises raw time traces of the one or more cerebral blood metrics.
Embodiment 46: The method of embodiment 30, wherein determining the representation of one or more cerebral blood metrics comprises extracting one or more features from the obtained information, wherein the one or more extracted features are provided as input to the computational model.
Embodiment 47: The method of embodiment 30, further comprising displaying the stroke risk score.
Embodiment 48: An apparatus for determining stroke risk, the apparatus comprising: a headband configured to attach to a head of a user during operation; a plurality of light sources attached to the headband; a plurality of light detectors attached to the headband; and one or more processors. The one or more processors configured to, during operation: cause, using the plurality of light sources, light to be emitted into a head of the user; obtain, using the plurality of light detectors, information indicative of light reflected from one more structures within the head of the user, wherein at least a portion of the obtained information is obtained during a time period of hypercapnic stimulation; based on the obtained information, and determine a stroke risk score.
Embodiment 49: The apparatus of embodiment 48, wherein the one or more processors are further configured to display the stroke risk score.
Embodiment 50: The apparatus of embodiment 48, wherein the one or more processors are further configured to determine the stroke risk score by: causing a determination of a representation of one or more cerebral blood metrics based on the obtained information; causing the representation of the one or more cerebral blood metrics to be provided as input to a computational model; and determining the stroke risk score based on output of the computational model.
Embodiment 51: The apparatus of embodiment 48, wherein the one or more processors are further configured to display the stroke risk score.
Embodiment 52: The apparatus of embodiment 48, wherein the plurality of light detectors comprises a camera.
Embodiment 53: The apparatus of embodiment 52, wherein the obtained information comprises a speckle pattern obtained using images captured by the camera.
Embodiment 54: The apparatus of embodiment 53, wherein the one or more cerebral blood metrics comprise a cerebral blood flow determined based on the speckle pattern.
Embodiment 55: The apparatus of embodiment 48, wherein the plurality of light sources comprises at least two light sources configured to emit light in different wavelengths.
Embodiment 56: The apparatus of embodiment 55, wherein the at least two light sources comprise a laser configured to emit light in an infrared wavelength range, and a light emitting diode (LED) configured to emit light in a near infrared wavelength range.
Embodiment 57: The apparatus of embodiment 48, wherein a distance between a light source of the plurality of light sources and a light detector of the plurality of light detectors is adjustable by changing a position of the light source and/or a light detector on the headband.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A method of determining stroke risk, the method comprising:
causing, using one or more light sources disposed on a headset worn by a user, light to be emitted into a head of the user;
obtaining, using one or more light detectors disposed on the headset, information indicative of light reflected from one more structures within the head of the user, wherein at least a portion of the obtained information is obtained during a time period of hypercapnic stimulation;
based on the obtained information, determining a representation of one or more cerebral blood metrics;
providing the representation of the one or more cerebral blood metrics as input to a computational model; and
determining a stroke risk score based on output of the computational model.

2. The method of claim 1, wherein the stroke risk score corresponds to a likelihood the user will experience a stroke over a predetermined future time period.

3. The method of claim 1, wherein the computational model is a trained machine learning model.

4. The method of claim 3, wherein the trained machine learning model comprises a deep neural network or a random forest classifier.

5. The method of claim 1, wherein another portion of the obtained information is obtained during a baseline time period before or after the time period of hypercapnic stimulation.

6. The method of claim 1, wherein the stroke risk score is an integer on a scale.

7. The method of claim 1, wherein during at least a portion of the time period of hypercapnic stimulation the user was (i) holding their breath or (ii) administered air with a concentration of carbon dioxide (CO₂).

8. The method of claim 1, wherein during at least a portion of the time period of hypercapnic stimulation the user was (i) holding their breath or (ii) administered air with a concentration of carbon dioxide (CO₂) of (a) less than 5%, (b) between 1% and 5%, or (c) between 1% and 10%.

9. The method of claim 1, further comprising:
determining an updated stroke risk score based on updated cerebral blood metrics;
determining a change between the stroke risk score and the updated stroke risk score; and
providing at least one recommendation based on the change between the stroke risk score and the updated stroke risk score.

10. The method of claim 1, wherein the one or more light sources comprise at least two light sources, each configured to emit light in a different wavelength.

11. The method of claim 1, wherein the one or more cerebral blood metrics comprise at least one of: cerebral blood flow, cerebral blood oxygenation, or cerebral blood volume, percentage change in cerebral blood flow.

12. The method of claim 11, wherein the cerebral blood flow is determined based on a decorrelation time associated with a series of speckle patterns obtained from a series of images captured by the one or more light detectors.

13. The method of claim 1, wherein the representation of one or more cerebral blood metrics comprises a ratio of percentage change in cerebral blood flow from a peak during the time period of hypercapnic stimulation to a baseline cerebral blood volume during a baseline time period.

14. The method of claim 1, wherein the representation of the one or more cerebral blood metrics comprises:
(i) raw time traces of the one or more cerebral blood metrics; or
(ii) extracted features from the obtained information.

15. The method of claim 1, further comprising displaying the stroke risk score.

16. An apparatus for determining stroke risk, the apparatus comprising:
a headband configured to attach to a head of a user during operation;
a plurality of light sources attached to the headband;
a plurality of light detectors attached to the headband; and
one or more processors configured to, during operation:
cause, using the plurality of light sources, light to be emitted into a head of the user;
obtain, using the plurality of light detectors, information indicative of light reflected from one more structures within the head of the user, wherein at least a portion of the obtained information is obtained during a time period of hypercapnic stimulation;
based on the obtained information, determine a stroke risk score; and
display the stroke risk score.

17. The apparatus of claim 16, wherein the one or more processors are further configured to display the stroke risk score.

18. The apparatus of claim 16, wherein the one or more processors are further configured to determine the stroke risk score by:
causing a determination of a representation of one or more cerebral blood metrics based on the obtained information;
causing the representation of the one or more cerebral blood metrics to be provided as input to a computational model; and
determining the stroke risk score based on output of the computational model.

19. The apparatus of claim 16, wherein:
the plurality of light detectors comprises a camera; and
the obtained information comprises a speckle pattern obtained using images captured by the camera.

20. The apparatus of claim 16. wherein the plurality of light sources comprises at least two light sources configured to emit light in different wavelengths.
